(19) United States
(12) Patent Application Publication
(10) Pub. No.: US 2016/0139129 A1
(43) Pub. Date: May 19, 2016

(54) BIOMARKERS FOR THE DIAGNOSIS AND THE RESPONSE TO TREATMENT OF PANCREATIC CANCER

(71) Applicants: UNIVERSIDAD DE GRANADA, Granada (ES); SERVICIO ANDALUZ DE SALUD, Sevilla (ES); UNIVERSIDAD DE JAÉN, Jaén (ES)

(72) Inventors: Carolina Torres Perales, Granada (ES); Sonia Perales Romero, Granada (ES); Juan Ramón Delgado Pérez, Granada (ES); Antonio Irigoyen Medina, Granada (ES); María José Alejandre Pérez, Granada (ES); José Iglesias Gómez, Granada (ES); Rogelio Palomino Morales, Granada (ES); Octavio Caba Pérez, Jaén (ES); José Carlos Prados Salazar, Granada (ES); Antonia Aránega Jiménez, Granada (ES); Ana Linares Gil, Granada (ES)

(21) Appl. No.: 14/898,254

(22) PCT Filed: Jun. 13, 2014
(86) PCT No.: PCT/ES2014/070495

§ 371 Date: Dec. 14, 2015

(30) Foreign Application Priority Data
Jun. 14, 2013 (ES) P201330896
Jun. 14, 2013 (ES) P201330897

(51) Int. Cl. G01N 33/574; C07K 16/24; C07K 16/28; C07K 16/22

(52) U.S. Cl. CPC G01N 33/57438; C07K 16/22; C07K 16/24; C07K 16/248; C07K 16/28; C07K 16/243; G01N 2333/50; G01N 2333/522; G01N 2333/5412; G01N 2333/47; G01N 2333/52

(57) ABSTRACT

The invention related to biomarkers, a method and a kit for early diagnosis of pancreatic cancer, in particular of pancreatic ductal adenocarcinoma (PDAC); and to biomarkers, a method and a kit or device for predicting or prognosticating an individual's response to combination treatment with a nucleoside analogue (preferably gemcitabine) and with a growth factor receptor (preferably erlotinib) in patients with pancreatic ductal adenocarcinoma.

### FIELD OF INVENTION

This invention is within the field of Molecular Biology and Medicine. Specifically, it relates to a method for obtaining data useful for the early diagnosis of ductal adenocarcinoma of the pancreas. Early diagnosis is performed by analysis of biomarkers FGF-10, CXCL11, OSM, GPNMB and SCF. It also relates to a method for obtaining data useful for predicting or prognosticating response to combined treatment with a nucleoside analogue (gemcitabine) with a growth receptor factor (erlotinib) in patients with ductal adenocarcinoma of the pancreas. The prognosis is performed by analysis of serum biomarkers CD80, EG-VEGF, IL-29, NRG1-beta1 and PD-ECGF.

### BACKGROUND OF THE INVENTION

### Ductal Adenocarcinoma of the Pancreas

Ductal adenocarcinoma of the pancreas (DACP), though it only accounts for 2.68% of all cancers, has one of the highest mortality rates among all types of cancer. The new cases of this cancer reached 44,000 individuals last year (2012) and 37,400 of them died (85%). Men and women have approximately a similar risk of suffering it (Siegel et al., 2012. C.A. Cancer J. Clin. 62, 10-29).

The reasons for low treatment response include late diagnosis, as in most cases when cancer is detected metastasis and intrinsic mechanisms of resistance to chemotherapy have occurred (Hidalgo, 2010. N. Engl. J. Med. 362, 1605-1617; Sakar et al. 2007. Toxicol. Appl. PharmacoL 224, 326-336). Due to the impact of early diagnosis in patient survival (Agarwal et al., 2008. Pancreas 36, 15-20), better diagnostic tools are required for detection and evaluation of the disease. Serum is the best option to obtain samples where to study malignant tumours as it can be taken by non-invasive methods. In the case of cancer of the pancreas it is particularly useful, as access to the target organ is very difficult. For these reasons, biomarkers of cancer based on serum are the easiest screening tests. To date, the only biomarker based on serum for DACP used is carbohydrate antigen 19-9 (CA), with sensitivity ranging from 70 to 90% and specificity from 70 to 98%, depending on the tumour size (Chan et al., 2012. J. Proteomics). However, this biomarker is not detected if tumour size is under 2 centimetres and for lack of expression in 10% of the patients. This also occurs in other diseases.

Therefore, since recently the American Society of Oncology does not recommend CA 19-9 for DACP screening tests (Duffy et al., 2010. Ann. Oncl. 21, 441-447). There is a close relationship between inflammatory conditions and carcinogenesis (Mantovani et al., 2008. Nature 454, 436-444; Germano et al., 2008. Cytokine 43, 374-379). Cancer of the pancreas is characterised by a dense desmoplastic reaction, which represents a major barrier that prevents effective release of chemotherapy agents at the main site of the disease. In addition, the tumour complex microenvironment nourishes invasion and metastasis by cancer of the pancreas (Shields et al., 2012. Biochem J. 441, 541-542; Nesse et al., 2011. Gut. 60, 861-868; Luo et al., 2012. Biochim. Biophys Acta. 1826, 170-178). Cytokines released by cancer cells or by cells inside the tumour microenvironment are the architects of this reaction. Based on the above, we consider that a modulation of cytokine levels could reflect the processes leading to the development of the disease or chemoresistance. Antibody-based arrays represent a useful tool for the discovery of cancer biomarkers. This method is remarkable for its capacity to provide fast, adequate perceptions for early detection of cancer biomarkers, providing new approaches for therapies against cancer (Breman et al., 2010. Nat. Rev. Cancer 10, 605-617). Therefore, the identification and validation of individual biomarkers or sets of biomarkers for fast detection of DACP (diagnostic biomarkers) would help increase patient survival.

On the other hand, the prognosis for patients with this disease is discouraging. Less than 5% of the patients reach 5 years of survival after the diagnosis due to the very low effect of chemotherapy in the treatment of disease and the metastatic condition of the tumour at the time of diagnosis. The disease is developed through a poorly known complex process, that covers a mix of mutations and multiple disorders in the cell signalling pathways. All of this explains the heterogeneity of this condition and the differences observed in the results between the different patients. In recent years no improvements have occurred to increase survival of patients with DACP (Hidalgo, 2010. Pancreatic cancer. N. Engl. J. Med. 362, 1605-1617; Costello et al., 2012. Nat. Rev. Gastroenterol Hepatol. 9, 435-444.)

### Biomarkers

A biological marker (biomarker), according to NIH Biomarker, is a characteristic that can be objectively measured and evaluated as an indicator of normal biological processes, pathological conditions or of pharmacological responses after a therapeutic procedure (Fong et al., 2012. Cancer J. 18, 530-538.).

In DACP three types of biomarkers are useful: those helping to detect the disease (diagnostic biomarkers); those predicting the response to treatments (predictive biomarkers) and those predicting the most probable course of the disease, including survival and recurrence pattern (prognostic biomarkers). The prognostic biomarkers can guide the treatment plants, helping to identify patients that could benefit from more aggressive interventions, new chemotherapy regimens and also help to establish new expectations for physicians and patients. In recent years, multiple investigations have been performed on biomarkers for the prognosis of DACP using immunohistochemistry, Western Blot, CRP, mRNA, proteomics and methods of methylation of DNA (Jamieson et al., 2011. Clin Cancer Res 17, 3316-333; Garcea et al., 2005. Eur. J. Cancer 41, 2213-2236; Luo et al., 2013. Hum. Pathol. 44, 69-76; Mann et al., 2012. PLoS One 7, e51119; Dallol et al., 2012. Cancer Epidemiol. Biomarkers. Prev. 21, 2069-2075).

This study was focused on the inflammatory mediators that could be useful biomarkers for the prognosis of patients with DACP even before they are treated. Disorders have been found in inflammatory cytokine levels of patients with cancer, even in early stages of development. The immune response plays a significant role during carcinogenesis, and circulatory inflammatory markers can be useful biomarkers for the diagnosis and prognosis of cancer (Schetter et al., 2010. Carcinogenesis 31, 37-49; Germano et al., 2008. Cytokine 43, 374-379). Cytokines are signalling molecules and act as key mediators of inflammation or of immune response. The starting hypothesis is based on the essential role of the microenvironment and the desmoplastic reaction in the development and progression of DACP so the pattern of expression of cytokines inside the tumour and the surrounding microenvironment could be potential prognostic biomarkers for DACP.

The objective of the study was to investigate the prognostic power of serum cytokines in response to the tumour in patients with DACP. To identify the most significantly adequate combination of biomarkers a conditional algorithm was used, based on a probabilistic analysis, adjusted to the Cox regression model. An equation was obtained for specific survival in this cohort.

### SHORT DESCRIPTION OF THE INVENTION

### Early Diagnosis Biomarkers

A first aspect of the invention relates to the use of genes (and/or proteins) FGF-10, CXCL11, OSM, GPNMB and SCF, for the early diagnosis of cancer of the pancreas.

The use can be simultaneous or any of the genes (and/or proteins), or any of the combination thereof can be selected.

In a preferred embodiment of this aspect of the invention the cancer of the pancreas is ductal adenocarcinoma of the pancreas (DACP)

Another aspect of the invention relates to a method for obtaining useful data, hereinafter the first method of the invention, for the early diagnosis of cancer of the pancreas, that comprises:
a) Obtaining an isolated sample from the individual.
b) Measuring the product of expression of genes that are selected from the list consisting of: FGF-10, CXCL11, OSM, GPNMB and SCF.

The product of expression of all genes can be measured simultaneously or any of the genes (and/or proteins), or any of the combinations thereof can be chosen.

In another preferred embodiment, the method of the invention also comprises:
c) comparing the amounts obtained in step (b) with a reference amount.

In a preferred embodiment of the method of the invention, steps (b) and/or (c) the methods described above can be completely or partially automated.

Another aspect of the invention relates to a diagnostic method for cancer of the pancreas that comprises steps (a)-(c) according to any of claims 3-5, and also comprises assigning the individual of step (a) to the group of individuals suffering cancer of the pancreas when they show an amount of expression of product of genes FGF-10, CXCL11, OSM, GPNMB and SCF, higher than the reference amount.

In a preferred embodiment of this aspect of the invention the cancer of the pancreas is ductal adenocarcinoma of the pancreas (DACP)

In a preferred embodiment of the method of the invention, the isolated biological method from an individual of step (a) is a blood sample.

In another preferred embodiment, the detection of the amount of expression of any of genes FGF-10, CXCL11, OSM, GPNMB and SCF is performed by immunoassay.

In another preferred embodiment, the immunoassay is an Enzyme-Linked ImmunoSorbent Assay (ELISA).

Another aspect of the invention relates to an antibody to treat an individual suffering cancer of the pancreas (or alternatively to the use of an antibody for the preparation of a medicine for the treatment of cancer of the pancreas), that can be identified by a method of the invention, wherein the antibody is selected from anti-FGF-10, anti-CXCL11, anti-OSM, anti-GPNM and anti-SCF, or any of its combinations. Preferably a combination of all the above antibodies is used. In another preferred embodiment of this aspect of the invention, the cancer of the pancreas is ductal adenocarcinoma of the pancreas (DACP).

Another aspect of the invention relates to a pharmaceutical composition that comprises a modulatory agent of at least one of genes that are selected from FGF-10, CXCL11, OSM; GPNMB and SCF, to treat an individual that suffers cancer of the pancreas (or alternatively to the use of a pharmaceutical composition that comprises a modulatory agent of at least one of genes that are selected from FGF-10, CXCL11, OSM, GPNMB and SCF in the preparation of a medicine for the treatment of cancer of the pancreas), and more preferably of ductal adenocarcinoma of the pancreas (DACP), that can be identified by a method of the invention.

Another aspect of this invention relates to a kit or device, hereinafter “first kit of the invention”, that comprises the elements necessary to measure the amount of products of expression of genes that are selected from FGF-10, CXCL11, OSM, GPNMB and SCF or any of its combinations.

In a preferred embodiment, the first kit of this invention comprises the antibodies that are selected from the list consisting of antibodies: anti-FGF-10, anti-CXCL11, anti-OSM, GPNM and anti-SCF, or any of its combinations. Preferably, the kit of the invention comprises simultaneously at least one antibody of each type: anti-FGF-10, anti-CXCL11, anti-OSM, anti-GPNM and anti-SCF.

In another preferred embodiment, the first kit of the invention comprises secondary antibodies or positive and/or negative controls. The kit can also include, with no type of limitation, buffers, protein extraction solutions, agents for preventing contamination, protein degradation inhibitors, etc.

Another aspect of the invention relates to a solid support, or protein chip, that comprises at least one of the antibodies anti-FGF-10, anti-CXCL11, anti-OSM, anti-GPNM and anti-SCF, or any of its combinations, to perform any of the methods of the invention.

Another aspect of the invention relates to a solid support, or DNA chip, that comprises oligonucleotides or single-channel microarrays designed from a known sequence or a mRNA of at least one of genes FGF-10, CXCL11, OSM, GPNM and SCF. Preferably, it comprises oligonucleotides that can detect the mRNA of all genes FGF-10, EG-VEGF, IL-29, NRG1-beta1 and PD-ECGF.

### Treatment Response Biomarkers

Another aspect of the invention relates to the use of genes (and/or proteins) CD80, EG-VEGF, IL-29, NRG1-beta1 and PD-ECGF, to predict or prognosticate the response of an individual to combination treatment with a nucleoside analogue plus an epidermal growth factor receptor inhibitor, wherein the individual suffers cancer of the pancreas.

In a preferred embodiment, in the treatment the nucleoside analogue is gemcitabine. In another preferred embodiment, in the treatment the epidermal growth factor receptor inhibitor is erlotinib.

In another preferred embodiment, the cancer is ductal adenocarcinoma of the pancreas (DACP).

Another aspect of the invention relates to a method for obtaining useful data, hereinafter third method of the invention, to predict or prognosticate the response of an individual to the treatment of cancer of the pancreas, that comprises:
a) Obtaining an isolated sample from the individual.
b) measuring the product of expression of genes that are selected from the list consisting of: CD80, EG-VEGF, IL-29, NRG1-beta1 and PD-ECGF.

In another preferred embodiment, the third method of the invention also comprises:
c) finding a prognostic index value (PI) with the values of cytokines of step b), applying the formula:

IP_{PDAC}=4.351×B7-1/CD80+0.003×EG-VEGF/PK1+0.081×IL-29+0.020×NGR1-beta1/HRG1-beta1+0.264×PD-ECGF

Where the individuals with PI>17 show a poor prognosis of the disease. In a preferred embodiment of this aspect, the individuals with PI>17 show a survival of less than five months.

In another preferred embodiment, individuals with PI≦17 show a better prognosis of the disease. In another preferred embodiment of this aspect, individuals with PI≦17 show a survival longer than five months.

In a preferred embodiment of the third method of the invention, the nucleoside analogue is gemcitabine. In another preferred embodiment, the epidermal growth factor receptor inhibitor is erlotinib.

In another preferred embodiment, the cancer is ductal adenocarcinoma of the pancreas (DACP).

In another preferred embodiment of this aspect of the invention, the biological sample (a) is a blood sample, and more preferably serum.

In another preferred embodiment, the detection of the amount of expression of any of genes CD80, EG-VEGF, IL-29, NRG1-beta1 and PD-ECGF is performed by an immunoassay.

In another preferred embodiment, the immunoassay is an Enzyme-Linked ImmunoSorbent Assay (ELISA).

Another aspect of the invention relates to an antibody for treating an individual that suffers cancer of the pancreas, that can be identified by a method of the invention, wherein the antibody is selected from anti-CD80, anti-EG-VEGF, anti-IL-29, anti-NRG1-beta1 and anti-PD-ECGF, or any of its combinations,

Another aspect of the invention relates to a pharmaceutical composition that comprises a modulatory agent of at least one of genes that are selected from CD80, EG-VEGF, IL-29, NRG1-beta1 and PD-ECGF, to treat an individual that suffers cancer of the pancreas (or alternatively to the use of a pharmaceutical composition that comprises a modulatory agent of at least one of the genes that are selected from CD80, EG-VEGF, IL-29, NRG1-beta1 and PD-ECGF in the preparation of a medicine for the treatment of cancer of the pancreas), that can be identified by a method of the invention.

Another aspect of this invention relates to a kit or device, hereinafter second kit of the invention, that comprises the elements necessary to measure the amount of products of expression of genes that are selected from CD80, EG-VEGF, IL-29, NRG1-beta1 and PD-ECGF or any of its combinations.

Even more preferably, the second kit of this invention comprises the antibodies that are selected from the list consisting of antibodies: anti-CD80, anti-EG-VEGF, anti-IL-29, anti-NRG1-beta1 and anti-PD-ECGF, or any of its combinations. Preferably, the second kit of the invention comprises simultaneously at least one antibody of each type: anti-CD80, anti-EG-VEGF, anti-IL29, anti-NRG1-beta1 and anti-PD-ECGF.

Another aspect of the invention relates to a solid support, or protein chip, that comprises at least one of the anti-CD80, anti-EG-VEGF, anti-IL-29, anti-NRG1-beta1 and anti-PD-ECGF antibodies, or any of its combinations or any of its combinations, to perform any of the methods of the invention.

Another aspect of the invention relates to a solid support, or DNA chip, that comprises oligonucleotides or single-channel microarrays designed from a known sequence or a mRNA of at least one of genes CD80, EG-VEGF, IL-29, NRG1-beta1 and PD-ECGF.

### Implementation of the Methods of the Invention

Another aspect of the invention relates to a computer-readable storage medium or a transmissible signal that comprises software instructions that can lead a computer to perform steps of any of the methods of the invention.

### DESCRIPTION OF INVENTION

In the description and the claims the word “comprises” and its variants do not intend to exclude other technical characteristics, additives, components or steps. For the experts in the matter, other objects, advantages and characteristics of the invention will be concluded in part of the description and in part of the practice of the invention.

### DEFINITIONS

An “isolated biological sample” includes, amongst others, cells, tissues and/or biological fluids of a body, obtained by any method known by an expert in the art.

The term “individual”, as used in the description, relates to animals, preferably mammals, and more preferably, humans. The term “individual” does not intend to be limiting in any aspect, and this can be of any age, gender and physical condition.

The expression levels of genes will give a specific profile of genetic expression. The term “expression level”, also called “amount of gene product” or “amount of expression product” relates to the biochemical material, wither RNA or protein, result of the expression of a gene. Sometimes a measure of the amount of gene product is used to conclude how active a gene is. “Genetic expression profile” is defined as the genetic profile obtained after measurement of the mRNA and/or protein produced by the genes of interest of biomarkers, that is, by the genes FGF-10, CXCL11, OSM, GPNMB, SCF, CD80, EG-VEGF, IL-29, NRG1-beta1 and PD-ECGF, in an isolated biological sample. The gene expression profile is performed, preferably, determining the levels of mRNA derived from transcription, after extraction of the total RNA present in the isolated biological sample, which can be performed through protocols known in the state of the art.

The detection of the amount of expression product of FGF-10, CXCL11, OSM, GPNMB, SCF, CD80, EG-VEGF, IL-29, NRG1-beta1 and PD-ECGF can be performed by any means known in the state of the art. The authors of this invention have shown that the detection of the amount or the concentration of antibodies against these cytokines on a semi-quantitative or quantitative basis allow to diagnose early individuals suffering ductal adenocarcinoma of the pancreas.

The measurement of the amount or concentration of expression product of these genes, preferably on a semi-quantitative or quantitative basis, can be performed directly or indirectly. The direct measure relates to the measurement of the amount or concentration of the product of expression of genes, based on a signal that is obtained directly from the transcripts of these genes or of the proteins, and that it is related directly to the number of RNA molecules or proteins produced by genes. This signal—that can be also referred to as signal of intensity—can be obtained, for instance, measuring a value of intensity of a chemical or physical property of said products. The indirect measurement includes the measure obtained from a secondary component or a biological measurement system (for instance, the measurement of cell response, ligands, “label” or enzyme reaction products).

The term “amount”, as used in the description, relates to, but not limited, the absolute or relative amount of the products of expression of genes or to the amount of antibodies, as well as to any value or parameter related to them or that can be derived from these. These values or parameters comprise values of signal of intensity obtained from any of the physical or chemical properties of aid products of expression obtained by direct measure. In addition, said values or parameters include all those obtained by indirect measurement, for instance, any of the systems of measurement described in another part of this document.

The term “comparison”, as used in the description, refers to but is not limited to, the comparison of the amount of products of expression of genes or of the biological sample to be analysed, also called test biological sample, with an amount of products of expression of genes of one or several desired reference samples. The reference sample may be analysed, for instance, simultaneously or consecutively, together with the test biological sample.

The term “marker compound”, as used in this description, relates to a compound that can lead to a chromogenic, fluorogenic, radioactive and/or chemoluminiscent signal which allows detection and quantitation of the amount of antibodies against FGF-10, CXCL11, OSM, GPNMB, SCF, CD80, EG-VEGF, IL-29, NRG1-beta1 and PD-ECGF. The marker compound is selected from the list that comprises radioisotopes, enzymes, fluorophores or any molecule susceptible of being conjugated with another molecule or detected and/or measured directly. This marker compound can bind to the antibody directly or through another compound. Some examples of marker compounds binding directly are, though not limited to, enzymes such as alkaline phosphatase or peroxidase, radioactive isotopes such as ³²P or ³⁵S, fluorochromes such as fluorescein or metal particles, for direct detection by colourimetry, auto-radiography, fluorometry or metallography respectively.

The term “immunoassay”, as used in this description relates to any analytical technique that is based on the reaction of conjugation of an antibody with an antigen. Examples of known immunoassays in the state of the art are, for instance, but not limited to: immunoblot, Enzyme-Linked ImmunoSorbent Assay (ELISA), lineal immunoassay (LIA), radioimmunoassay (RIA), immunofluorescence, x-map or protein chips.

The terms “polynucleotide” and “nucleic acid” are used herein exchangeably, referring to polymer forms of nucleotides of any length, both ribonucleotides (RNA) and deoxyribonucleotides (DNA).

The terms “amino acid sequence”, “peptide”, “oligopeptide”, “polypeptide” and “protein” are used herein exchangeably, and relate to a polymer form of amino acids of any length, that can be coding or non-coding, chemically or biochemically modified.

### Early Diagnosis Biomarkers

The authors of this invention have analysed the family members of cytokines in healthy individuals, in untreated individuals with DACP and in individuals suffering DACP that have been subject to treatment with gemcitabine and erlotinib. A number of markers have been found that allow the early diagnosis of individuals with DACP. Therefore, this invention provides a method for obtaining useful data for the early diagnosis of individuals with DACP.

Therefore, a first aspect of the invention relates to the use of genes (and/or proteins) FGF-10, CXCL11, OSM, GPNMB and SCF, for the early diagnosis of cancer of the pancreas. The use can be simultaneous or any of the genes (and/or proteins), or any the combination thereof can be selected.

In a preferred embodiment of this aspect of the invention the cancer of the pancreas is ductal adenocarcinoma of the pancreas (DACP)

Another aspect of the invention relates to a method for obtaining useful data, hereinafter the first method of the invention, for the early diagnosis of cancer of the pancreas, that comprises:
a) Obtaining an isolated sample from the individual.
b) Measuring the product of expression of genes that are selected from the list consisting of: FGF-10, CXCL11, OSM, GPNMB and SCF.

The product of expression of all genes can be measured simultaneously or any of the genes (and/or proteins), or any of the combinations thereof can be chosen.

In another preferred embodiment, the first method of the invention also comprises:
c) comparing the amounts obtained in step (b) with a reference amount.

In a preferred embodiment, steps (b) and/or (c) of the methods described above can be completely or partially automated, for instance, by means of a sensor robotic equipment for the detection of the amount in step (b) or the computerised comparison in step (c).

A preferred embodiment of this aspect of the invention relates to a diagnostic method for cancer of the pancreas that comprises steps (a)-(c) of the first method of the invention, and also comprises assigning the individual of step (a) to the group of individuals suffering cancer of the pancreas when they show an amount of expression products of genes FGF-10, CXCL11, OSM, GPNMB and SCF, higher than the reference amount. An amount of expression product of any of genes FGF-10, CXCL11, OSM, GPNMB and SCF, or all of them simultaneously, above the reference amount, can occur.

In a preferred embodiment the method of the invention, the isolated biological sample of an individual of step (a) is a blood sample, and even more preferably serum.

The comparison described in section (c) of the method of this invention can be performed manually or computer-assisted.

The adequate reference amounts can be measured by the method of this invention from a reference sample that can be analysed, for instance, simultaneously or consecutively, together with the test biological sample. Therefore, for instance, but not limited to, the reference sample can be the negative controls, that is, the amounts detected by the method of the invention in samples of individuals not suffering the disease.

The characteristics of the cytokines that are object of evaluation are described below:

The gene FGF-10 or fibroblast growth factor 10 (KGF-2: keratinocyte growth factor 2) is in chromosome 5 (5p13-p12). This factor has been described as a promoter of morphogenesis in primary stages of organogenesis as well as a regulator of pancreatic epithelial stem cell proliferation (Bhushan et al., 2001. Development 128, 5109-5117). A link has been also recently described between signalling FGF10/FGFR2-IIIb and migration and invasion of pancreatic cancer cells through induction of metalloproteinases of type 1 membrane matrix and transforming the growth factor TGF-β1 which is an important regulator of mesenchymal epithelial transition (Nomura et al., 2008. Br. J. Cancer 99, 305-313).

In this invention, FGF-10 is also defined by a nucleotide or polynucleotide sequence, forming the coding sequence of the protein included in SEQ ID NO: 8, and that would comprise several variants from:

a) nucleic acid molecules coding a polypeptide that comprises the amino acid sequence of SEQ ID NO: 8,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 8, and wherein the polypeptide coded by said nucleic acids has the activity and the structural characteristics of the FGF-10 protein. These nucleic acid molecules include that of sequence SEQ ID NO: 1.

The gene CXCL11 or hemokine (C-X-C motif) ligand 11 (C-X-C motif chemokine 11; beta-R1; interferon gamma-inducible protein 9; interferon-inducible T-cell alpha chemoattractant; small inducible cytokine B11; small inducible cytokine subfamily B (Cys-X-Cys), member 11; small inducible cytokine subfamily B (Cys-X-Cys), member 9B; small-inducible cytokine B11, H174, I-TAC, PI-9, IP9, SCYB11, SCYB9B, b-R1) is in chromosome 4 (4q21.2). CXCL11 is a chemokine that interacts specifically with the receptor CXCR3. It stimulates phosphorylation of the MARK kinase pathways leading to proliferation and prevention of apoptosis (Miekus et al., 2010. Folia Histochem. Cytobiol. 48, 104-111) In addition, its role has been described in several cancer tumorigenesis (Lo et al., 2010. Am. J. Pathol. 176, 2435-2446; Furuya et al., 2011. Gynecol. Oncot 122, 648-655). Although we report herein, for the first time, CXCL11 as a possible biomarker in patients with DACP, it has been recently proposed as a serum biomarker for adenocarcinoma of the prostate (Klee et al., 2012. Clin, Toxicol, 58, 599-609)

In this invention, CXCL11 is also defined by a nucleotide or polynucleotide sequence, forming the coding sequence of the protein included in SEQ ID NO: 9, and that would comprise several variants from:

a) nucleic acid molecules coding a polypeptide that comprises the amino acid sequence of SEQ ID NO: 9,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 9, and wherein the polypeptide coded by said nucleic acids has the activity and the structural characteristics of the CXCL11 protein. These nucleic acid molecules include that of sequence SEQ ID NO: 2.

The gene OMS or oncostatin M (C-X-C motif chemokine 11; beta-R1; interferon gamma-inducible protein 9; interferon-inducible T-cell alpha chemoattractant; small inducible cytokine B11; small inducible cytokine subfamily B (Cys-X-Cys), member 11; small inducible cytokine subfamily B (Cys-X-Cys), member 9B; small-inducible cytokine B11, H174, I-TAC, PI-9, IP9, SCYB11, SCYB9B, b-R1) is contained in chromosome 4 (4q21.2). Oncostatin M belongs to the same family as interleukin-6 and stimulates several functions involved in wound repair. Although it was initially described as a leukaemia cell growth inhibitor (Zarling et al, 1986. Proc. Nat. Acad. Sci. USA. 83, 9739-43), it has been recently seen that it can have a double role, also as promoter in cell proliferation, migration and invasion (Fossey et al 2011, BMC Cancer 11, 125; Li et al., 2011. Int. J. Mol. Med. 28, 101-108; Winder et al., 2011. J. Pathol. 225, 448-462; Tiffen et al., 2008. Mol. Endocrinol. 22, 2677-2688). Once the OMS binds its receptor, it promotes mutual phosphorylation and activation of the Janus kinase family pathway (JAK)/STAT. Several transcriptional objectives of STAT3 are important contributing factors to the biology of DACP (Corcoran et al., 2011. Cancer Res. 71, 5020-5029)

In this invention, OMS is also defined by a nucleotide or polynucleotide sequence, forming the coding sequence of the protein included in SEQ ID NO: 10, and that would comprise several variants from:

a) nucleic acid molecules coding a polypeptide that comprises the amino acid sequence of SEQ ID NO: 10,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 10, and wherein the polypeptide coded by said nucleic acids has the activity and the structural characteristics of the OMS protein. These nucleic acid molecules include that of sequence SEQ ID NO: 3.

The gene GPNMB or glycoprotein (transmembrane) nmb (UNQ1725/PRO9925, HGFIN, NMB, glycoprotein NMB; glycoprotein nmb-like protein; osteoactivin; transmembrane glycoprotein HGFIN; transmembrane glycoprotein, NMB NQ1725/PRO9925, HGFIN, NMB) is contained in chromosome 7 (7p15). Glycoprotein GPNMB, also known as osteoactivin (OA), DC-HIL or HGFIN is a type 1 transmembrane protein, which was described in the beginning as low at undetectable levels in malignant cells (Weterman et al., 1995. Int. J. Cancer 60, 73-81) However, more recent studies have described GPNMB/osteoactivin as promoter of metastasis and invasion (Rose et al., 2010. PLos One 5, 12093) in some cancers such as melanoma (Tomihari et al., 2010. Cancer Res 70, 5778-5787; Tse et al., 2006. Clin. Cancer Res. 12, 1373-1382), uveal melanoma (Williams et al., 2010. Melanoma res. 20, 184-190), glioma (Kuan et al., 2006. Clin. Cancer Res. 12, 1970-1982), hepatocellular carcinoma (Onaga et al., 2003. J. Hepatol. 39, 779-785 and breast cancer, where it has been also described as a prognostic indicator of recurrence (Rose et al., 2010. Clin. Cancer Res. 16, 2147-2156). An induction of GPNMB in the bone marrow of patients with amyotrophic lateral sclerosis as inducer of motor neuron degeneration has been recently described (Tanaka et al., 2012. Sci. Rep. 2, 573).

In this invention, GPNMB is also defined by a nucleotide or polynucleotide sequence, forming the coding sequence of the protein included in SEQ ID NO: 11 or of SEQ ID NO: 12, and that would comprise several variants from:

a) nucleic acid molecules coding a polypeptide that comprises the amino acid sequence of SEQ ID NO: 11 or of SEQ ID NO: 12,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 11 or of SEQ ID NO: 12, and wherein the polypeptide coded by said nucleic acids has the activity and the structural characteristics of the GPNMB protein. These nucleic acid molecules include that of sequence SEQ ID NO: 4 or of SEQ ID NO: 5.

The gene SCF or KIT ligand (KITLG, FPH2, KL-1, Kitl, MGF, SCF, SF, SHEP7, c-Kit ligand; familial progressive hyperpigmentation 2; kit ligand; mast cell growth factor; steel factor; stem cell factor) is contained in chromosome 12 (12q22). SCF is the main ligand for the tyrosine kinase receptor c-kit (KIT). Its bindings support proliferation, differentiation and survival in cells expressing KIT, both in normal cells and in tumour cells, including pancreatic cancer cells (Yasuda et al., 2006. Mol. Cancer 5, 46) through activation of pathways (PI3K)/Akt; MARK and STAT (Yasuda et al., 2007. Dig. Dis. Sci. 52, 2292-2300). Previous analyses have described the high levels of stem cell factor (SCE) in colorectal cancer sera and DACP (Mroczko et al., 2005. Clin. Toxicol. Lab. Med. 43, 146-150; Mroczko et al., 2004. Clin. Toxicol. Lab. Med. 42, 256-260; Mroczko et al., 2005. Int. J. Colorectal Dis. 22, 33-38).

In this invention, SCF is also defined by a nucleotide or polynucleotide sequence, forming the coding sequence of the protein included in SEQ ID NO: 13 or of SEQ ID NO: 14, and that would comprise several variants from:

a) nucleic acid molecules coding a polypeptide that comprises the amino acid sequence of SEQ ID NO: 13 or of SEQ ID NO: 14,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 13 or of SEQ ID NO: 14, and wherein the polypeptide coded by said nucleic acids has the activity and the structural characteristics of the SCF protein. These nucleic acid molecules include that of sequence SEQ ID NO: 6 or of SEQ ID NO: 7.

As DACP shows multiple genetic and epigenetic disorders and implicates several signalling pathways, a combination of CA 19-9 with several biomarkers could represent a new approach to establish new diagnostic biomarkers. FGF-10, CXCL11, OSM, GPNMB and SCF have been selected by the authors of this invention for an early diagnosis of the disease.

In another preferred embodiment, the detection of the amount of expression of any of genes FGF-10, CXCL11, OSM, GPNMB and SCF is performed by an immunoassay.

In another preferred embodiment, the immunoassay is an Enzyme-Linked ImmunoSorbent Assay (ELISA). The ELISA is based on the assumption that an immunoreagent (antigen or antibody) can be immobilised in a solid support, then placing this system in contact with a fluid phase containing the complementary reagent that can be bound to a marker compound. There are different types of ELISA: Direct ELISA, indirect ELISA, or sandwich ELISA:

Another aspect of the invention relates to an antibody to treat an individual suffering cancer of the pancreas (or alternatively to the use of an antibody for the preparation of a medicine for the treatment of cancer of the pancreas), that can be identified by a method of the invention, wherein the antibody is selected from anti-FGF-10, anti-CXCL11, anti-OSM, anti-GPNM and anti-SCF, or any of its combinations. Preferably a combination of all the above antibodies is used.

Another aspect of the invention relates to a pharmaceutical composition comprising an agent modulating at least one of the genes that are selected from FGF-10, CXCL11, OSM; GPNMB and SCF, to treat an individual suffering cancer of the pancreas (or alternatively to the use of a pharmaceutical composition comprising an agent modulating at least one of the genes that are selected from FGF-10, CXCL11, OSM; GPNMB and SCF in the preparation of a medicine for the treatment of cancer of the pancreas), that can be identified by a method of the invention. Preferably, the composition comprises one or more agents modulating all genes FGF-10, CXCL11, OSM; GPNMB and SCF simultaneously. Even more preferably, the pharmaceutical composition also comprises another active ingredient. In another preferred embodiment, the modulatory agent is an antibody that is selected from anti-FGF-10, anti-CXCL11, anti-OSM, anti-GPNM and anti-SCF, or any of its combinations.

Another aspect of this invention relates to a kit or device, hereinafter first kit of the invention, that comprises the elements necessary to measure the amount of products of expression of genes that are selected from FGF-10, CXCL11, OSM, GPNMB and SCF or any of its combinations.

In a preferred embodiment, the first kit of this invention comprises the antibodies that are selected from the list consisting of antibodies: anti-FGF-10, anti-CXCL11, anti-OSM, anti-GPNM and anti-SCF, or any of its combinations. Preferably, the kit of the invention comprises simultaneously at least one antibody of each type: anti-FGF-10, anti-CXCL11, anti-OSM anti-GPNM and anti-SCF.

In another preferred embodiment, the first kit of the invention comprises secondary antibodies or positive and/or negative controls. The kit can also include, with no type of limitation, buffers, protein extraction solutions, agents for preventing contamination, protein degradation inhibitors, etc.

On the other hand, this kit can include all supports and containers necessary for set up and optimisation. Preferably, this kit comprises also the instructions to perform the methods of the invention.

Another aspect of the invention relates to a solid support, or protein chip, that comprises at least one of the antibodies anti-FGF-10/, anti-CXCL11, anti-OSM, anti-GPNM and anti-SCF, or any of its combinations, to perform any of the methods of the invention.

Another aspect of the invention relates to a solid support, or DNA chip, that comprises oligonucleotides or single-channel microarrays designed from a known sequence or a mRNA of at least one of genes FGF-10, GXCL11, OSM, GPM and SCE Preferably, it comprises oligonucleotides that can detect the mRNA of all genes FGF-10, EG-VEGF, IL-29, NRG1-beta1 and PD-ECGF.

Therefore, for instance, the oligonucleotides sequences are built in the chip surface by elongation of a chain in growth with a single nucleotide using photolithography. Therefore, oligonucleotides are anchored in edge 3′ by a method of selective activation of nucleotides, protected by a photolabile reagent, by selective incidence of light through a photomask. The photomask can be physical or virtual.

Therefore, oligonucleotide probes can be from 10 to 100 nucleotides, more preferably, from 20 to 70 nucleotides, and even more preferably, from 24 to 30 nucleotides. For measuring gene expression, preferably about 40 oligonucleotides per gene are used.

Synthesis in situ on a solid support (for instance, glass) could be performed by ink-jet technology, which requires longer probes. Supports could be, amongst others, filter or membranes of NC or nylon (charged), silicon or glass slides for microscopes covered with aminosilanes, polylysine, aldehydes or epoxy. The probe is each of the chip samples. The target is the sample to be analysed. Messenger RNA, total RNA, CRP fragment, etc.

### Treatment Response Biomarkers

On the other hand, the authors of this invention have analysed 507 proteins of the family of cytokines in the serum of individuals suffering DACP and that have been treated with the combination gemcitabine+erlotinib. A number of markers have been found which allow the prognosis of individuals with DACP. Therefore, this invention provides a method for obtaining useful data that prognosticate the response to treatment with gemcitabine+erlotinib of individuals with DACP, even before they are subject to said treatment.

Therefore, another aspect of the invention relates to the use of genes CD80, EG-VEGF, IL-29, NRG1-beta1 and PD-ECGF, to predict or prognosticate the response of an individual to treatment with a nucleoside analogue and an epidermal growth factor receptor inhibitor, wherein the individual suffers cancer of the pancreas.

In a preferred embodiment, the nucleoside analogue is gemcitabine. In another preferred embodiment, the epidermal growth factor receptor inhibitor is erlotinib.

In another preferred embodiment, the cancer is ductal adenocarcinoma of the pancreas (DACP).

Another aspect of the invention relates to a method for obtaining useful data, hereinafter third method of the invention, to predict or prognosticate the response to treatment of cancer of the pancreas, that comprises:
a) Obtaining an isolated sample of serum from the individual.
b) measuring the product of expression of genes that are selected from the list consisting of: CD80, EG-VEGF, IL-29, NRG1-beta1 and PD-ECGF.

In another preferred embodiment, this method also comprises:
c) finding a prognostic index value (PI) with the values of cytokines of step b):

IP_{PDAC}=4.351×B7-1/CD80+0.003×EG-VEGF/PK1+0.081×IL-29+0.020×NGR1-beta1/HRG1-beta1+0.264×PD-ECGF

Where, the individuals with PI>17 show a poor prognosis of the disease, as survival is shorter than five months.

In another preferred embodiment, individuals with PI<17 show a better prognosis of the disease, as the survival is longer than five months.

In a preferred embodiment de this method, in the treatment the nucleoside analogue is gemcitabine. In another preferred embodiment, in the treatment the epidermal growth factor receptor inhibitor is erlotinib.

In another preferred embodiment, the cancer is ductal adenocarcinoma of the pancreas (DACP).

In another preferred embodiment of this aspect of the invention, the biological sample (a) is a blood sample.

Steps (b) and/or (c) described can be completely or partially automated, for instance, by means of a sensor robotic equipment for the detection of the amount in step (b) or the computerised comparison in step (c).

Preferably, the isolated biological sample of an individual of step (a) is a blood sample (serum).

The comparison described in section (c) of the method of this invention can be performed manually or computer-assisted.

The adequate reference amounts can be measured by the method of this invention from a reference sample that can be analysed, for instance, simultaneously or consecutively, together with the test biological sample.

The characteristics of the cytokines that are object of evaluation are described below:

CD80 (also known as B7-1): the B7 system is one of the most important secondary signalling mechanism and it is essential to maintain the delicate balance between the immune power and autoimmunity suppression. B7-1 (CD80) and B7-2 (CD86) are ligands in antigen-presenting cells and they are all responsible for c-stimulating signalling because the regulate growth, maturation and tolerance of the T cell (Seger et al., 2012. Cancer Immunol. Immunother. 61, 1327-1341). After binding of its receptors the T cells are activated and survival is promoted (Chen et al., 1994. J. Exp. Med. 179, 523-532). On the other hand they can also deliver co-inhibitory signals in the inhibitor receptors and block the response of the T cells. An inadequate co-stimulation could contribute to the progressive growth of tumours. The combination of B7-1 and of B7-H1 has been already proposed as a prognostic factor for DACP. Although the role of B7-1 appears to be anti-tumoral, there is a new global view, where it is believed that the aberrant or imbalanced expression of members of the family B7 could contribute to escape of immune control (Wang et al., 2012. PLoS One 7, e45491; Wang et al., 2010. World J. Surg. 34, 1059-1065).

In this invention, CD80 is also defined by a nucleotide or polynucleotide sequence, forming the coding sequence of the protein included in SEQ ID NO: 24, and that would comprise several variants from:

a) nucleic acid molecules coding a polypeptide that comprises the amino acid sequence of SEQ ID NO: 24,

b) nucleic acid molecules where the complementary hybrid chain with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 24, and wherein the polypeptide coded by said nucleic acids has the activity and the structural characteristics of the CD80 protein. These nucleic acid molecules include that of sequence SEQ ID NO: 15.

EG-VEGF (also known as PK1): this molecule was described in principle as an example of a type of specific mitogen to regulate proliferation and differentiation of vascular endothelium on a specific-tissue manner. Although this protein does not show any structural homology to the VEDF family, it has in common different regulating functions related to proliferation and migration (LeCouter et al., 2001. Nature 412, 877-884). It has been also described that EG-VEGF is related to cancer of the ovary (Balu et al., 2012. Rom. J. Morphol. Embryol. 53, 479-483), colorectal (Nagano et al., 2007 J. Surg. Oncol. 96, 605-610), prostate (Pasquali et al., 2006. Endocrinology 147, 4245-4251), hepatocellular (Li et al., 2006. J. Exp. Ciin. Cancer Res. 25, 403-409), pancreas (Jiang at al., 2009. Pancreatology 9, 165-172) and neuroblastoma (Ngan et al., 2007. Clin. Cancer Res. 13, 868-875), As well as a factor for angiogenesis of the placenta (Brouillet, S., 2012. Trends Endocrinol. Metab. 23, 501-508).

In this invention, EG-VEGF is also defined by a nucleotide or polynucleotide sequence, forming the coding sequence of the protein included in SEQ ID NO: 25, and that would comprise several variants from:

a) nucleic acid molecules coding a polypeptide that comprises the amino acid sequence of SEQ ID NO: 25,

b) nucleic acid molecules with the complementary chain hybrid with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 25, and wherein the polypeptide coded by said nucleic acids has the activity and the structural characteristics of the EG-VEGF protein. These nucleic acid molecules include that of sequence SEQ ID NO: 16.

IL-29: it is also named as IFN-λ1 and belongs to type III of the family IFN. It has been described to induce biological activities similar to those of type I of the same family. Although both can induce the anti-proliferative response in many types of cells, IFN-λI appears to be more limited. Signalling produced by IFN-λI triggers activation of the routes STAT1, STAT2, STAT3 and STAT5, that are the main 3 cascades of kinase proteins activated by mitogen (MARK) and the phosphorylation of protein kinase B (Akt) through the route phosphatidylinositol-3-kinase (PI3K; Kotenko et al., 2011. Curr. Opin. Immunol. 23, 583-590; Maher et al., 2008. Cancer Biol. Ther. 7, 1109-1115; Donnelly et al., 2010. J. Interferon Cytokine Res. 30, 555-564). However, the ability of IFN-λI to trigger these alternative routes, might be specified for altered cells or cancer cells. The conclusion results from other studies suggesting that the induction of multiple myeloma human cell growth occurs by activation of MARK (Novak et al., 2008. Leukemia 22, 2240-2246). The specific role of IL-29 in the response of the host and the immune vigilance has not been defined yet in the context of cancer in general and in the context of DACP in particular.

In this invention, IL-29 is also defined by a nucleotide or polynucleotide sequence, forming the coding sequence of the protein included in SEQ ID NO: 26, and that would comprise several variants from:

a) nucleic acid molecules coding a polypeptide that comprises the amino acid sequence of SEQ ID NO: 26,

b) nucleic acid molecules with the complementary chain hybrid with the polynucleotide sequence of a),

c) nucleic acid molecules where the sequence differs from a) and/or b) due to degeneration of the gene code,

d) nucleic acid molecules coding a polypeptide that comprises the amino acid sequence with an identity of at least 80%, 90%, 95%, 98% or 99% with SEQ ID NO: 26, and wherein the polypeptide coded by said nucleic acids has the activity and the structural characteristics of the IL-29 protein. These nucleic acid molecules include that of sequence SEQ ID NO: 17.

NRG1-beta1: neuregulin-1 or heregulin-1 is a ligand of an extracellular protein aimed at binding to members of the family of receptors ErbB, ErbB3 and ErbB4. After the interaction with its receptors several biological events are stimulated, including the induction and progression of some epithelial cancers. Proteins NRG1/HRG1 also play an essential role in the nervous system, the heart and the breast, and are involved in the development of some diseases, including schizophrenia and cancer of the breast (Falls et al., 2003. Exp. Cell. Res. 284, 14-30; Hayes at al., 2008. J. Mammary Gland. Biol. Neoplasia 13, 205-214). Regulation of the angiogenic factor VEGF by NRG1/HRG1 (Yen et al., 2000. Oncogene 19, 3460-3469) has been also described. Its proliferative effects are probably achieved through the combined action with multiple pathways, including PI3K, MARK and p38MARK pathways (Stove et al., 2004. Clin. Exp. Metastasis 21, 665-684) that have been specifically described in DACP cells. DACP patients with a worse survival rate had a high expression of HRG-β mRNA. ErbB3 plays an essential role in pancreatic carcinogenesis as it promotes proliferation of cancer cells both in vivo and in vitro. It has been recently seen how fibroblasts associated with cancer develop the ligand NRG1/FIRG1, activating DACP cells by signalling mediated by ErbB3/AKT and increasing carcinogenesis. This could be related to the insufficient effect of erlotinib (EGFR inhibitor) when it is combined with gemcitabine in the treatment of patients with DACP (Liles at al, 2011. Br. J. Cancer 105, 523-533).

In this invention, NRG1/HRG1 is also defined by a nucleotide or polynucleotide sequence, forming the coding sequence of the protein included in SEQ ID NO: 27, and that would comprise several variants from;

a) nucleic acid molecules coding a polypeptide that comprises the amino acid sequence of SEQ ID NO: 27,

b) nucleic acid molecules with the complementary chain hybrid with the polynucleotide sequence of a),

c) nucleic acid molecules where the sequence differs from a) and/or b) due to the degeneration of the genetic code,

d) nucleic acid molecules coding a polypeptide that comprises the amino acid sequence with an identity of at least 80%, 90%, 95%, 98% or 99% with SEQ ID NO: 27, and wherein the polypeptide coded by said nucleic acids has the activity and the structural characteristics of the NRG1/HRG1 protein. These nucleic acid molecules include that of sequence SEQ ID NO: 18.

PD-ECGF: also known as thymidine phosphorylase. Its activity and expression in carcinomas of the oesophagus, stomach, lung, pancreas and colorectal is significantly greater than in non-malignant adjacent tissues and may play a major role in the proliferation of these solid tumours. PD-ECGF is expressed both in tumour cells and in stromal cells associated to tumour. In the analyses of regression of carcinoma of the bladder, colorectal, gastric, renal and pancreas, PD-ECGF has been marked as a prognostic factor for poor results.

In this invention, PD-ECGF is also defined by a nucleotide or polynucleotide sequence, forming the coding sequence of the protein included in SEQ ID NO: 28, SEQ ID NO: 29, SEQ ID NO: 30, SEQ ID NO: 31, SEQ ID NO: 32, and that would comprise several variants from:

a) nucleic acid molecules coding a polypeptide that comprises the amino acid sequence of SEQ ID NO: 28, SEQ ID NO: 29, SEQ ID NO: 30, SEQ ID NO: 31, SEQ ID NO: 32

b) nucleic acid molecules with a complementary chain hybrid with the polynucleotide sequence of a),

c) nucleic acid molecules where the sequence differs from a) and/or b) due to the degeneration of the genetic code,

d) nucleic acid molecules coding a polypeptide that comprises the amino acid sequence with an identity of at least 80%, 90%, 95%, 98% or 99% with SEQ ID NO: 28, SEQ ID NO: 29, SEQ ID NO: 30, SEQ ID NO: 31, SEQ ID NO: 32, and wherein the polypeptide coded by said nucleic acids has the activity and the structural characteristics of the PD-ECGF protein. These nucleic acid molecules include that of sequence SEQ ID NO: 19, SEQ ID NO: 20, SEQ ID NO: 21, SEQ ID NO: 22, SEQ ID NO: 23.

In another preferred embodiment, the detection of the amount of expression of any of genes CD80, EG-VEGF, IL-29, NRG1-beta1 and PD-ECGF is performed by an immunoassay.

In another preferred embodiment, the immunoassay is an Enzyme-Linked ImmunoSorbent Assay (ELISA). The ELISA is based on the assumption that an immunoreagent (antigen or antibody) can be immobilised in a solid support, then placing this system in contact with a fluid phase containing the complementary reagent that can be bound to a marker compound. There are different types of ELISA: Direct ELISA, indirect ELISA, or sandwich ELISA:

Another aspect of the invention relates to an antibody to treat an individual that suffers cancer of the pancreas, that can be identified by a method of the invention, wherein the antibody is selected from anti-CD80, anti-EG-VEGF, anti-IL-29, anti-NRG1-beta1 and anti-PD-ECGF, or any of its combinations. Preferably a combination of ail the above antibodies is used.

Another aspect of the invention relates to a pharmaceutical composition that comprises a modulatory agent of at least one of the genes that are selected from CD80, EG-VEGF, IL-29, NRG1-beta1 and PD-ECGF, to treat an individual that suffers cancer of the pancreas (or alternatively to the use of a pharmaceutical composition that comprises a modulatory agent of at least one of the genes that are selected from CD80, EG-VEGF, IL-29, NRG1-beta1 and PD-ECGF in the preparation of a medicine for the treatment of cancer of the pancreas), that can be identified by a method of the invention. Preferably, the composition comprises one or more modulatory agents of all genes CD80, EG-VEGF, IL-29, NRG1-beta1 and PD-ECGF, simultaneously. More preferably, the cancer of the pancreas is ductal adenocarcinoma of the pancreas. Even more preferably, the pharmaceutical composition also comprises another active ingredient. In another preferred embodiment, the modulatory agent is an antibody that is selected from anti-CD80, anti-EG-VEGF, anti-IL-29, anti-NRG1-beta1 and anti-PD-ECGF, or any of its combinations.

Another aspect of this invention relates to a kit or device, hereinafter second kit of the invention, that comprises the elements necessary to measure the amount of products of expression of genes that are selected from CD80, EG-VEGF, IL-29, NRG1-beta1 and PD-ECGF or any of its combinations.

Even more preferably, the second kit of this invention comprises the antibodies that are selected from the list consisting of antibodies: anti-CD80, anti-EG-VEGF, anti-IL-29, anti-NRG1-beta1 and anti-PD-ECGF, or any of its combinations. Preferably, the second kit of the invention comprises simultaneously at least one antibody of each type: anti-CD80, anti-EG-VEGF, anti-IL29, anti-NRG1-beta1 and anti-PD-ECGF.

In another preferred embodiment, the second kit of the invention comprises secondary antibodies or positive and/or negative controls. The kit can also include, with no type of limitation, buffers, protein extraction solutions, agents for preventing contamination, protein degradation inhibitors, etc.

On the other hand, this kit can include all holders and containers necessary for set up and optimisation. Preferably, this kit comprises also the instructions to perform the methods of the invention.

In a preferred embodiment, the second kit of the invention comprises the assignment of a prognostic index value and classification of individuals, so that: if the individuals show PI>17 they have a poor prognosis of the disease, while if the individuals show PI<17 they have a better prognosis of the disease. Preferably, the individuals with PI≦17 show a survival longer than 5 months. In another preferred embodiment, the individuals with PI>17 show a survival shorter than 5 months.

Another aspect of the invention relates to a solid support, or protein chip, that comprises at least one of the antibodies anti-CD80, anti-EG-VEGF, anti-IL-29, anti-NRG1-beta1 and anti-PD-ECGF, or any of its combinations or any of its combinations, to perform any of the methods of the invention.

Another aspect of the invention relates to a solid support, or DNA chip, that comprises oligonucleotides or single-channel microarrays designed from a known sequence or a mRNA of at least one of genes CD80, EG-VEGF, IL-29, NRG1-beta1 and PD-ECGF. Preferably, it comprises oligonucleotides that can detect the mRNA of all genes CD80, EG-VEGF, IL-29, NRG1-beta1 and PD-ECGF.

Therefore, for instance, the oligonucleotides sequences are built in the chip surface by elongation of a chain in growth with a single nucleotide using photolithography. Therefore, oligonucleotides are anchored in edge 3′ by a method of selective activation of nucleotides, protected by a photolabile reagent, by selective incidence of light through a photomask. The photomask can be physical or virtual.

Therefore, oligonucleotide probes can be from 10 to 100 nucleotides, more preferably, from 20 to 70 nucleotides, and even more preferably, from 24 to 30 nucleotides. For measuring gene expression, preferably about 40 oligonucleotides per gene are used.

Synthesis in situ on a solid support (for instance, glass) could be performed by ink-jet technology, which requires longer probes. Supports could be, amongst others, filters or membranes of NC or nylon (charged), silicon or glass slides for microscopes covered with aminosilanes, polylysine, aldehydes or epoxy. The probe is each of the chip samples. The target is the sample to be analysed. Messenger RNA, total RNA, CRP fragment, etc.

### Implementation of the Methods of the Invention

Another aspect of the invention relates to a computer readable storage medium that comprises software instructions that can lead a computer to perform the steps of the method according to any of the methods of the invention.

Another aspect of the invention relates to a transmissible signal that comprises software instructions that lead a computer to perform steps of any of the methods of the invention.

### BRIEF DESCRIPTION OF THE FIGURES

FIG. 1 (A) shows the disease-specific Kaplan-Meier survival curves for the whole study population. The Kaplan-Meier survival curve is defined as the probability of surviving in a defined time period. Each time period is the time between two non-simultaneous terminal events. There were no censored survival data, as no information was lost on the survival time of any individual. (B-H) these graphs represent the Kaplan-Meier survival curves of each of the individual biomarkers, labelled as significant prognostic markers: (B), clinical response; (C) age; (D), BDNF; (E), HVEM/TNFRSF14; (F), IL-24; (G), IL-29; (H), leptin receptor; (I) LRP-6 and (J), ROBO4. For dichotomisation the most significant cut-off values in terms of survival were used. The p values for the log-rank test are shown for each variable.

FIG. 2. Cox regression model. Prognostic curves observed (shown by dots with square, triangle and diamond shape) and planned (shown as solid line) for patients with DACP according to (A): univariate model or (B): multivariate odds ratio model. With the Cox regression it is intended to detect any relationship between the risk that a given event studied (death) and one or more independent variables are detected. (B) The three curves corresponding to the models generated with 3, 4 and 5 cytokines are represented. As expected, the prognosis curves were adjusted to a logarithmic distribution. The coefficient of determination R² is illustrative of the goodness of fit model. As coefficient, these models provide useful predictions, the most accurate multivariate model being that of 5 cytokines, reaching 92.6%. Therefore, our PI model approaches 93% of the survival variation.

FIG. 3. PI Kaplan-Meier survival curves. (A) shows the survival graph for PI derived from the univariate model, covering 2 cytokines. 1.5 as chosen as cut-off value to divide the cohort of patients of short survival time (<5 months) and long survival time (>5 months). (B) shows the survival graph for PI derived from the multivariate model, covering the 5 cytokines. 17 was chosen as cut-off value to divide the cohort of patients of short survival time (<5 months) or long survival time (>5 months). The p values for the log-rank tests are shown for both comparisons.

FIG. 4 The analysis of ROC curves for each of the five serum cytokines evidenced that they were differentially expressed among patients with DACP. The ROC curves summarised the precision of the cytokines in the prediction of patients with DACP. The area under the curve (ABC) is the mean sensitivity of the biomarker. A biomarker with non-predictive values would have an AUC of 0.5, while a biomarker with a perfect capacity to predict the disease, would have an AUC of 1. A shows the AUC values, cut-off value, sensitivity and specificity for FGF-10; B shows the AUC values, cut-off value, sensitivity and specificity for I-CXCL11; C shows the AUC values, cut-off value, sensitivity and specificity for OSM; D shows the AUC values, cut-off value, sensitivity and specificity for GPNMB; E shows the AUC values, cut-off value, sensitivity and specificity for SCF; F shows the AUC values, cut-off value, sensitivity and specificity for five combined cytokines. The cut-off values (intensity signal values) selected were those with both the highest sensitivity and specificity.

### EXAMPLES OF THE INVENTION

The following examples and drawings are provided by way of illustration, and it is not intended that they limit this invention.

All the data and statistical analysis was performed using the software IBM SPSS statistic 20 or with statistical language R. The quality of the analysis was increased using the package “ArrayQualityMetrics” in R to remove any possible atypical value.

### Early Diagnosis Biomarkers

### Materials and Methods

Thirty-nine patients participated in the study. Three different groups were established:
1) Twelve healthy patients, as control group.
2) Fourteen patients with DACP that had received no treatment (called pre-treated).
3) Thirteen patients with DACP under 2 weeks of treatment with the combination therapy gemcitabine+erlotinib (called post-treated)

All the patients were diagnosed with DACP at the Hospital Virgen de las Nieves (Granada, Spain) between 2008 and 2011. All patient information, including gender, age, degree of disease, and symptoms, was recorded. The mean age of the patients was 66 years (range 41-79 years) and male-female ratio 50:50. The clinical classification of the patients with adenocarcinoma of the pancreas was: state III (28%) and state IV (72%; Table 1).

TABLE 1
Clinical-pathological characteristics of the study population
(n = 14)

| | |
|---|---|
| Age at diagnosis, years (mean ± SD) | 66 ± 10.5 |
| Gender | M: 50% |
| | F: 50% |
| Disease stage | III (28%) |
| | IV (72%) |
| Type of chemotherapy | Gemcilabine |
| | + |
| | Erlotinib |
| Clinical response | PR (14.29%) |
| | SD (21.43%) |
| | PD (64.29%) |
| Survival time, months (mean ± SD) | 12.6 ± 12.6 |
| CEA levels [μg/l] (mean ± SD) | 2219 ± 5017 |
| | Healthy: |
| | 0-37 |
| CA level 19-9 [U/l] (mean ± SD) | 899 ± 3185 |
| | Healthy: |
| | 0-5 |

PR: partial response;
SD: stable disease;
PD: progressive disease;
SD: standard deviation.

Table 1. Clinical-pathological characteristics of the study population (n=14). Information gathered from all DACP patients participating in the study.

### Source of the Samples.

The blood samples were collected after obtaining approval from the relevant ethics committees and consents signed by the patients. A total of 39 serum samples were collected from 2009 to 2011, using the standard procedures of the Oncology Department of the Hospital Virgen de las Nieves (Granada, Spain). The blood samples were obtained from patients diagnosed with DACP at the start of the study and 2 weeks after the start of therapy (gemcitabine+erlotinib) and also in healthy individuals. The serum was obtained after blood centrifugation at 1500 rpm for 10 minutes at 4° C. Aliquots of the samples were taken and stored at −80° C.

### Cytokine Antibody Assay

The proteins soluble in serum from patients with DACP were measured using a biotin label-based antibody array (Human Antibody L-series 507 Array (RayBiotech, Norcross, Ga., USA), according to the recommended protocols. In short, the samples were biotinised. The antibodies were immobilised in given sites in glass slide. Incubation of the arrays with biological samples consisted of cytokines binding to their relevant antibodies. The signals are viewed using conjugated streptavidin-HRP and colourimetry. The final intensities were measured as the original intensities except for the background. The data were normalised to the positive control for each individual result

### Statistical Analysis

As the cytokines in all groups did not show a normal distribution, the Mann-Whitney test was used to highlight the differences between groups (p<0.05). In addition the fold change (FC) was calculated. The fold change values of cytokines were tested to indicate the relative expression values. Neither FC≦1.5 nor FC≧1.5 in the intensity signal between the groups were considered relevant. The area under the curve (AUC) of the receptor operative characteristic (ROC) of each marker was calculated to evaluate its diagnostic significance representing the rate of true positive values (sensitivity) vs the rate of false positive values (1-specificity). In addition, a marker combination index was generated and the ROC curve was also drawn for this combination. The cut-off values were selected to improve both sensitivity and specificity. In addition, box plots were represented that show the modulation of the expression of common cytokines modified significantly between post-treated vs pre-treated and between pre-treated and control.

### Results

Analysis of Serum Diagnosis Biomarkers in Patients with DACP and Healthy (Control).

For the purpose of finding markers helping to the early detection of DACP, an extensive screening was performed, with an antibody array for 507 human cytokines to identify those cytokines expressed differentially in health individuals and those suffering DACP. The clinical-pathological characteristics of the patients with DACP are summarised in Table 1. As shown in Table 2, there are five over-expressed cytokines in patients with DACP not treated when compared to healthy volunteers (p<0.05). The cytokines FGF-10, CXCL11, OSM, GPNMB and SCF could represent an altered serum profile in patients with DACP.

TABLE 2
Cytokines significantly over-expressed in patients with DACP.

| ID | P-Value | Log FC | FC |
|---|---|---|---|
| FGF-10 | 0.040 | 1.10 | 2.15 |
| CXCL11 | 0.046 | 0.93 | 1.91 |
| OSM | 0.040 | 0.96 | 1.95 |
| GPNMB | 0.019 | 1.36 | 2.56 |
| SCF | 0.022 | 1.51 | 2.85 |

Table 2. The 5 cytokines significantly over-expressed. The differences were obtained by the Mann-Whitney test (p<0.05). The relative expression levels are given by the corresponding FC. Each FC≧1.5 or ≦1/1.5 in the intensity of signal between groups was considered relevant.

### Analysis of Sensitivity and Specificity of Serum Biomarkers for Early Diagnosis of DACP.

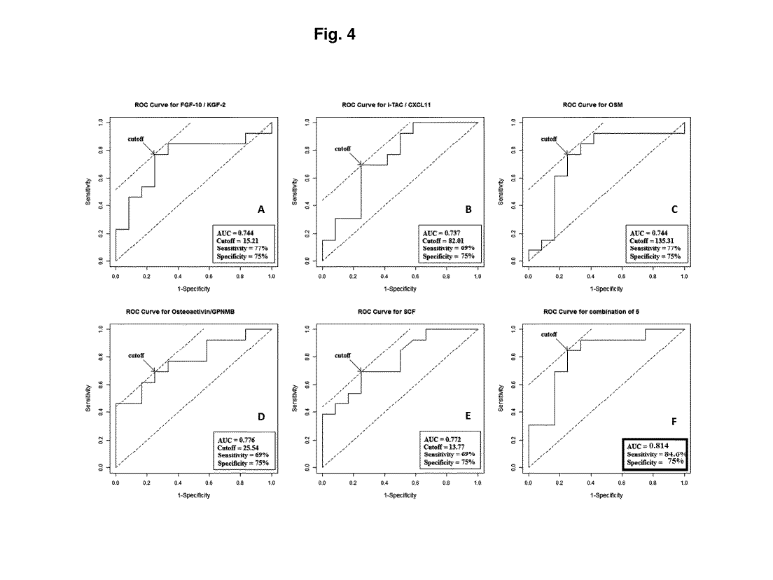

To establish whether these 5 cytokines could be used as markers for early detection of DACP, the diagnostic value (the ability to distinguish between health and disease) of each biomarker, alone and in combination, was evaluated. The sensitivity and specificity for each biomarker were analysed using the method of the area under the curve of the operative characteristics of the receptor (ROC). The curves are based on cytokine levels in healthy individuals and individuals before and after treatment. Each candidate biomarker was investigated independently (FIG. 4A-E). All possible new markers had AUC values from 0.74 to 0.78 to distinguish healthy from DACP patients. The areas under the curve ROC for cytokines FGF-10, CXCL11, OSM, GPNMB and SCF were 0.744, 0.737, 0.744, 0.776 and 0.772 respectively. Osteoactivin stood out for developing the highest sensitivity for prediction of DACP. Then, to establish if these 5 cytokines overall could be used as group of biomarkers for the detection of DACP, a combination of these 5 markers was analysed to obtain an integrated ROC curve. The combined group of these cytokines improved significantly the ability of all biomarkers separately alone to distinguish patients with cancer of the pancreas of healthy controls, the AUC value improved to 0.841 (FIG. 4F).

To establish the sensitivity and specificity of each biomarker, the cut-off points providing the best waived points between the higher sensitivity and the higher specificity (FIG. 4). All had a specificity of 75% and a sensitivity with a range within 69-77%. The group of the 5 cytokines evidence the best yield to detect DACP in serum samples, also with a sensitivity of 77% but a specificity of 83%, higher than separately.

### Treatment Response Biomarkers

All study patients were diagnosed with DACP at the Hospital Virgen de las Nieves (Granada, Spain) between 2008 and 2011. All patient information was recorded: gender, age, degree of disease, and symptoms. The mean age of the patients was 66 years (range 41-79 years) and male-female ratio (50:50). The clinical classification of the patients with adenocarcinoma of the pancreas was: state III (28%) and state IV (72%; Table 3). The overall survival time of the patients with DACP was 12.6 months and were treated with combination therapy with +erlotinib. The blood samples were collected once approval was obtained from the relevant ethics committees and consent from the donors. The serum samples were collected between 2009 and 2011, using standard procedures at the oncology department of the Hospital Virgen de las Nieves. In addition, the blood samples were obtained from patients diagnosed with DACP at the baseline conditions in patients after 2 weeks after the start of therapy (gemcitabine+erlotinib) and also in healthy individuals. The serum was obtained after blood centrifugation at 1500 rpm for 10 minutes at 4° C. The samples were aliquoted and stored at a temperature of −80° C.

Clinical-pathological characteristics of the study population (n = 14)

| | |
|---|---|
| Age at diagnosis, years (mean ± SD) | 66 ± 10.5 |
| Gender | M: 50% |
| | F: 50% |
| Disease stage | III (28%) |
| | IV (72%) |
| Type of chemotherapy | Gemcitabine |
| | + |
| | Erlotinib |
| Clinical response | PR (14.29%) |
| | SD (21.43%) |
| | PD (64.29%) |
| Survival time, months (mean ± SD) | 12.6 ± 12.6 |
| CEA levels [μg/l] (mean ± SD) | 2219 ± 5017 |
| | Healthy: |
| | 0-37 |
| CA level 19-9 [U/l] (mean ± SD) | 899 ± 3185 |
| | Healthy: |
| | 0-5 |

PR: partial response;
SD: stable disease;
PD: progressive disease;
SD: standard deviation.

Table 3. Clinical-pathological characteristics of the study population (n=14) Information compiled from all DACP patients participating in the study

### Cytokine Antibody Assay

A human biotin-based antibody array (Human Antibody L-series 507 array (RayBiotech, Norcross, Ga., USA)) was used, and according to the protocols recommended to measure proteins soluble in sera of patients with DACP. All samples were biotinised. The antibodies were immobilised at specific sites of the glass slide. The array membranes were incubated with the biological samples to give rise to binding of cytokines with their corresponding antibodies. The signals were viewed using conjugated streptavidin-HRP and colorimetry. The intensities of the final points were calculated as the original intensity, subtracting from the bottom. The data were normalised to the positive controls in the individual device.

### Statistical Analysis

The mean survival after administration of gemcitabine and erlotinib was calculated in months from the start of treatment to death. For an analysis of overall survival (S), the Kaplan-Meier was used a method to estimate the probability of survival in a given time, using the percentage of patients surviving that time. The log rank test was used to establish the survival differences between groups. The Kaplan-Meier survival curves for individual markers were obtained after dichotomisation. The cut-off values for each marker were those developing the most significant survival between the two groups. To establish the most significant variables contributing to OS, univariate and multivariate analyses were performed with the Cox regression model of odds ratio to establish associations between serum cytokines and cancer-related mortality. First, the association between mortality and cytokine levels was analysed, considered one factor every time. Then the Cox odds ratio multivariate model was applied, using a stepwise conditional algorithm, based on the probability rate.

The global model adjustment was considered to be significant according to the Chi-squared test. (p<0.05). In addition, the Wald index was shown to measure the weight of each variable in the global model, both in univariate and multivariate. The levels of 507 cytokines were entered in the models, as continuous parameters, and the results were expressed as a hazard ratio or relative risk ratio. By the analysis of this variables, the prognostic index (PI) was obtained, which considers the regression coefficients derived from the Cox model of all significant factors. The differences were considered significant when p<0.05.

The Cox regression model for the study of DACP patient survival was defined by the following equation:

$$h ( t ) = {h}_{0} ( t ) {e}^{{\sum }_{j = 1}^{N} {β}_{i j} {x}_{i j}} \qquad Ecuación 1$$

Where h(t) is the hazard ratio at time t, hₒ (t) is the baseline hazard ratio and the exponent, which does not depend on time, is known as prognostic index (PI). The prognostic index for a specific patient is defined as:

$${PI}_{i} = {\sum }_{j = 1}^{N} {β}_{i j} {x}_{i j} \qquad Equation 2$$

Where βᵢⱼ defines the coefficient planned for the Cox regression model for a particular patient i and cytokine j; xᵢⱼ determines the expression level measured for the patient i and cytokine j; and N represents the number of cytokines included in the model.

### Results

Analysis of Survival of Patients with DACP

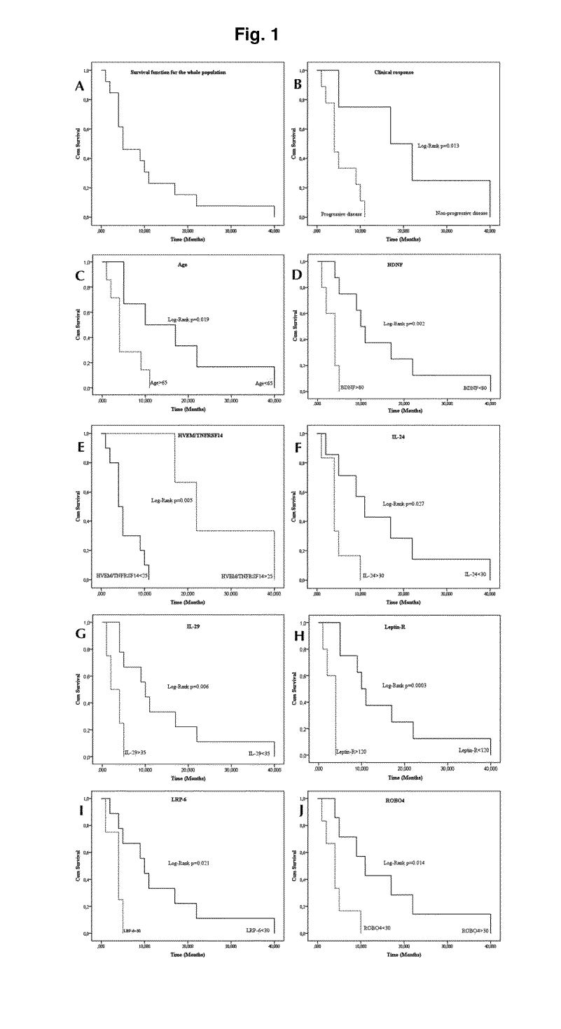

The clinical characteristics of the DACP patients are summarised in Table 1. For the whole of the study population, the OS rates were 46.15% at 6 months, 23.08% at 12 months and 7.69% at 24 months. The mean duration of the follow-up for the study group was 12 months (range 1-40 months) and during this time 100% of the patients with DACP died due to the disease. The probabilities of survival were calculated using the Kaplan-Meier method. The survival curve for the entire patient cohort is shown in FIG. 1A.

### Univariate Analysis Between Serum Eytokines and Survival

First, a univariate approach was used in the study to identify the measurable diagnostic factors that were relevant and independent and that could be associated with a high risk of death for DACP. Serum levels of cytokines and the clinical-pathological parameters such as age, gender, state and clinical response were analysed. The clinical-pathological parameters, age and clinical response (progressive or non-progressive disease) had a low weight in the prognosis according to the univariate analysis (p=0.030 and p=0.013, respectively). With regard to cytokines, the univariate analysis of the expression levels of BDNF (p=0.034, HR 1.005, 95% CI (1.000-1.009)); HVEM/TNFRSF14 (p=0.039, HR 0.924, 95% CI (0.858-0.996)); IL-24 (p=0.023 HR 1.041, 95% CI (1.006-1.078)); IL-29 (p=0.021, HR 1.012, 95% CI (1.002-1.023)); Leptin R (p=0.018, HR 1.008, 95% CI (1.001-1.015)); LRP-6 (p=0.022 HR 1.027, 95% CI (1.004-1.051)) and ROBO4 (p=0.045 HR 1.002, 95% CI (1.000-1.004)) had a significant influence in the prognosis. The results of the univariate analysis of each cytokine, and of the prognostic factors independently, and its beta-coefficients, hazard ratio (HR) represents the factor for which the risk changes for each unit that increases the expression of cytokines), 95% CI (upper and lower limits of the confidence interval with a significant level of 0.05) and p values, are shown in Table 4.

TABLE 4
Prognostic factors according to the univariate analysis

| | Overall survival | | | | |
|---|---|---|---|---|---|
| Variable | β | HR | 95% Cl | | p-value |
| BDNF | 0.005 | 1.005 | 1.000 | 1.009 | 0.034 |
| HVEM/TNFRSF14 | −0.079 | 0.924 | 0.858 | 0.996 | 0.038 |
| IL-24 | 0.040 | 1.041 | 1.006 | 1.078 | 0.023 |
| IL-29 | 0.012 | 1.012 | 1.002 | 1.023 | 0.021 |
| Leptin R | 0.008 | 1.008 | 1.001 | 1.015 | 0.018 |
| LRP-6 | 0.027 | 1.027 | 1.004 | 1.051 | 0.022 |
| ROBO4 | 0.002 | 1.002 | 1.000 | 1.004 | 0.045 |
| Age. | 0.086 | 1.089 | 1.008 | 1.177 | 0.030 |
| Clinical response | 2.064 | 8.706 | 1.057 | 71.692 | 0.013 |
| | Fit of the general model (p = 0.0023) | | | | |
| Cytokines | β | HR | 95% Cl | | p-value |
| II-24 (1) | 0.042 | 1.042 | 1.003 | 1.023 | 0.026 |
| II-29 (2) | 0.014 | 1.014 | 1.005 | 1.081 | 0.017 |

β: coefficient according to the Cox regression model for a given patient and cytokine;
HR: Random ratio (represents the factor whereby the hazard ratio changes for each unit of increase of cytokine expression);
95% Cl: upper and lower limit of the confidence interval with a significance level of 0.05.

Table 4. Prognostic factors according to the univariate analysis Variables significantly associated with survival of patients with DACP in the univariate analysis. The following are shown: The beta β)-coefficients, hazard ratio, 95% CI and p values for the variables selected. The positive beta-coefficients for an explanatory variable represent a high risk and, therefore, the prognosis is worse. On the contrary, a negative regression coefficient involves a better prognosis for patients with higher values of this variable. The hazard ratio is a descriptive measure used to compare the survival times of the two different groups of patients. The hazard ratio indicates the change in the risk of death if the variable increases by one unit (an unit of expression for cytokines, one year for the age variable and disease progression in clinical response). 95% CI is the confidence interval for the hazard ratio. The p values, in the top of the table, show the significance of each individual variable explaining the survival. The model is shown stepwise in the bottom of the table, using only significant cytokines according to the univariate analysis. The p values indicated show the significance of cytokines in the complete model.

For the purpose of establishing the survival differences of these markers individually in patients with DACP, Kaplan-Meier survival curves were generated using the cut-off points, providing the most significant discrimination in terms of survival between groups. FIG. 1 (B-J) shows the Kaplan-Meier survival group of individual markers that show significant differences in the prognosis.

### Multivariate Analysis Between Serum Cytokines and Survival

Although statistically significant variables of the univariate analysis are often included in the multivariate analysis, some variables that are not significant in the univariate analysis could appear as significant in the multivariate analysis. Furthermore, in addition to the statistically significant variables related to the prognosis in the univariate analysis, those without impact were also included. The best combination of the cytokines selected according to the Multivariate Cox hazard ratio is shown in Table 5.

TABLE 5
Prognostic factors according to the multivariate analysis

| | | | | | | Adjustment |
|---|---|---|---|---|---|---|
| | | | | | | of general |
| | Overall survival | | | | | model |
| Cytokines | β | HR | 95% Cl | | p-value | p-value |
| IL-29 | 0.081 | 1.084 | 1.010 | 1.164 | 0.026 | 0.004212 |
| B7-1/CD80 | 4.351 | 77.574 | 1.138 | 5289.453 | 0.043 | 0.002494 |
| PD-ECGF | 0.264 | 1.302 | 0.944 | 1.797 | 0.108 | 0.001350 |
| EG-VEGF/PK1 | 0.003 | 1.003 | 1.000 | 1.005 | 0.049 | 0.000134 |
| NRG1-beta1/ | 0.020 | 1.020 | 0.994 | 1.047 | 0.129 | 0.000286 |
| HRG1-beta1 | | | | | | |

β: coefficient according to the Cox regression model for a given patient and cytokine;
HR: Random ratio (represents the factor whereby the risk changes for each unit of increase of cytokine expression);
95% Cl: upper and lower limit of the confidence interval with a significance level of 0.05.

Table 5. Prognostic factors according to the multivariate analysis: The cytokines significantly associated with survival of patients with DACP in the multivariate analysis: The following are shown: coefficients-beta (β), hazard ratio (HR), 95% CI and p values for cytokines selected. The last column of p values represents the significance of cytokines in the complete model.

None of the clinical-pathological parameters demonstrated a significant trend to the reduction of overall survival (p>0.05) and were not considered in the global model. Referring to cytokines and according to the multivariate analysis, the expression levels of CD80 (p=0.043, HR 77,574, 95% CI (1.138-5289.4)); EG-VEGF (p=0.049, HR 1.003, 95% CI (1.000-1.005)) and IL-29 (p=0.026, HR 1.084, 95% CI (1.010-1.164)), had a significant influence in the prognosis. The significant influence of IL-29 in survival was seen in the univariate analysis and was confirmed with the multivariate analysis. The beta (β)-coefficients, hazard ratio (HR), 95% CI and p values for cytokines selected are shown in Table 3. Although NRG1-beta1 ((p=0.129), HR 1.020, 95% CI 0.994-1.797)) did not show a significant influence in the prognosis as independent factor.

Prognosis Indices of Cytokines in Patients with DACP:

An adequate combination of biomarkers could provide us with the most accurate information on the prognosis; therefore, the most accurate set of variables was searched for using the conditional regression protocol based on the odds ratio.

To illustrate the inter-related effect in OS of the 7 markers remarked by the univariate analysis, the Cox odds ratio was used for selecting variables related jointly with survival. As a result of this analysis, a model resulted that contained only IL-24 (p=0.026, HR 1.042, 95% CI, (1.003-1.023)) and IL-29 (p=0.017, HR 1,014, 95% CI (1.005-1.081)). The global adjustment fit was shown to be significant for the statistical Chi-squared test (p=0.0023). For the purpose of establishing a prognostic index to establish overall survival of the patients with DACP, these beta-coefficients of cytokines were entered in the following equation and the following prognostic index model was generated:

PIᵤₙᵢᵥₐᵣᵢₐₜₑ=0.042×IL-24+0.014×IL-29

The univariate PI represents the multivariate model derived from the combination of significant markers in the univariate analysis. With regard to the cytokines obtained by multivariate analysis, a second statistically significant survival model (p=0.0003) was built, and the following PI model was generated:

PIₘᵤₗₜᵢᵥₐᵣᵢₐₜₑ=4.351×B7-1/CD80+0.003×EG-VEGF/PK1+0.081×IL-29+0.020×NGR1-beta1/HRG1-beta1+0.264×PD-ECGF

The multivariate PI represents the multivariate model derived from the best of all possible combinations using the 507 cytokines of the multivariate analysis.

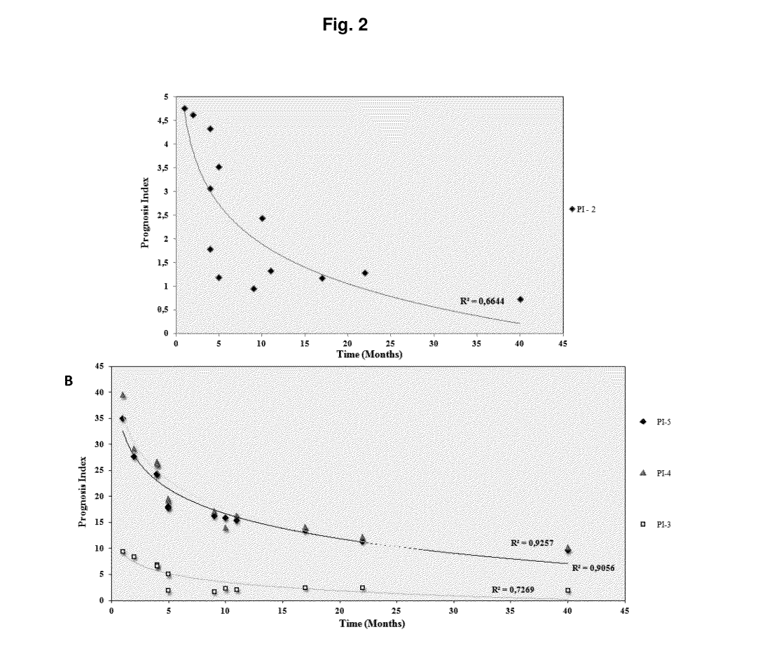

It was evaluated by regression analysis if the PI could generate an accurate survival model for this cohort of patients. The R² was used as evidence of goodness of fit. The results of the univariate PI and multivariate PI proposed were calculated, classified and related to OS. As expected, both survival models evidenced a logarithmic trend when represented vs time. FIG. 2 represents the results of the PI observed and the planned log-adjustments for these models. According to the multivariate model, the global model with 5 cytokines was statistically significant, though the regression analyses were also evaluated for the models containing 3 and 4 cytokines. The R² values obtained were 0.664, 0.727, 0.906 and 0.926 for univariate PI model and for the multivariate PI models with 3, 4 and 5, respectively. All models gave satisfactory results, but the multivariate model covering CD80, EG-VEGF, IL-29, NRG1-beta1 and PD-ECGF, stood out from the rest.

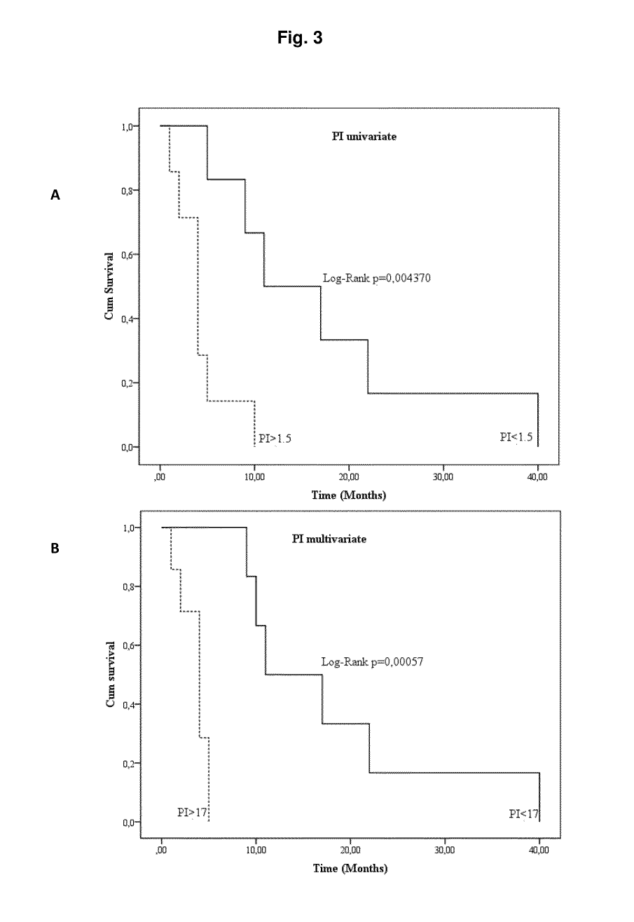

The prognosis index for the multivariate model with these 5 cytokines ranged from 0 to 40 in our cohort. The patients were categorised in two groups, according to their prognosis indices: unfavourable prognosis (PI>17) and favourable prognosis (PI<17). The survival curves were then compared between these two prognostic groups (FIG. 3 A). The proposed groups that are shown to distinguish the overall survival time are: low (<5 months) and high (>5 months). Overall survival in these groups was statistically very significant (p<0.00056). The prognostic index for univariate model was also represented, and its range was from 0 to 5. According to this PI, the patients were categorised again in 2 groups: unfavourable prognosis (PI>1.5) and favourable prognosis (PI<1.5). In addition, the survival curves were compared between these 2 prognostic groups (FIG. 3 B), and a significant correlation was obtained with the overall survival, as low survival (<5 months) and high survival 5 months). The overall survival in these groups was less, but still significant (p<0.004) compared to the multivariate PI.

### Clauses

In Summary, the Aspects of this Invention are Described in the Following Numbered clauses:

1.—The simultaneous use of genes: CD80, EG-VEGF, IL-29, NRG1-beta1 and PD-ECGF, to predict or prognosticate the response of an individual to the treatment comprising a nucleoside analogue and a epidermal growth factor receptor inhibitor, wherein the individual suffers cancer of the pancreas.

2.—The use according to the above clause, wherein the individual suffers ductal adenocarcinoma of the pancreas (DACP).

3.—The use, according to any of the above clauses, wherein the treatment comprises the nucleoside analogue gemcitabine and the growth factor receptor inhibitor erlotinib.

4.—A method for obtaining useful data, to predict or prognosticate the response of an individual to the treatment that comprises a nucleoside analogue and an epidermal growth factor receptor inhibitor, wherein the individual suffers cancer of the pancreas, that comprises:
a) obtaining an isolated biological sample from the individual.
b) measuring the amount of product of expression of genes: CD80, EG-VEGF, IL-29, NRG1-beta1 and PD-ECGF.

5.—The method according to the above clause, that also comprises:
c) finding a prognostic index (PI) value with the cytokine values of step b) applying the formula:

IP_{PDAC}=4.351×B7-1/CD80+0.003×EG-VEGF/PK1+0.081×IL-29+0.020×NGR1-beta1/HRG1-beta1+0.264×PD-ECGFC)

6. A method to predict or prognosticate the response of an individual to the treatment comprising a nucleoside analogue and an epidermal growth factor receptor inhibitor, wherein the individual suffers cancer of the pancreas, comprising steps (a)-(c) according to any of clauses 4-5, and also comprises classifying individuals with PI>17 in the group of individuals with a poor prognosis of the disease and preferably a survival shorter than 5 months.

7.—A method to predict or prognosticate the response of an individual the treatment comprising a nucleoside analogue and an epidermal growth factor receptor inhibitor, wherein the individual suffers cancer of the pancreas, comprising the steps (a)-(c) according to any of clauses 4-5, and also comprises classifying individuals with PI≦17 in the group of individuals with a better prognosis of the disease, and more preferably a survival over 5 months.

8.—The method according to any of clauses 4-7, wherein the nucleoside analogue is gemcitabine.

9.—The method according to any of clauses 4-8, wherein the epidermal growth factor receptor inhibitor is erlotinib.

10.—The method according to any of clauses 4-9, wherein the individual suffers ductal adenocarcinoma of the pancreas (DACP).

11.—The method according to any of clauses 4-10, wherein the biological sample is a blood sample (serum).

12.—The method according to any of clauses 4-11, where steps (b) and/or (c) the methods described above can be completely or partially automated.

13.—The method according to any of clauses 4-12, wherein the measurement is performed by immunoassay.

14.—The method according to any of clauses 4-13, wherein the immunoassay is an ELISA.

15.—The use of antibody in the preparation of a medicine to treat an individual that suffers cancer of the pancreas, that can be identified a method according to any of clauses 4-14, wherein the antibody is selected from anti-CD80, anti-EG-VEGF, anti-IL29, anti-NRG1-beta1 and anti-PD-ECGF, or any of its combinations.

16.—A pharmaceutical composition that comprises a modulatory agent of at least one of genes that are selected from CD80, EG-VEGF, IL-29, NRG1-beta1 and PD-ECGF, to treat an individual that suffers cancer of the pancreas, that can be identified by the method according to any of clauses 4-14.

17.—A kit or device, that comprises the elements necessary to measure the amount of expression of genes that are selected from CD80, EG-VEGF, IL-29, NRG1-beta1 and PD-ECGF.

18.—The kit or device, according to the above clause, that comprises the antibodies anti-CD80, anti-EG-VEGF, anti-NRG1-beta1 and anti-PD-ECGF.

19.—The kit or device according to clauses 17-18, that also comprises the assignment of a prognostic index value and classification of individuals, according to clauses 5-6.

20.—A solid support, or protein chip, that comprises at least one of the antibodies anti-CD80, anti-EG-VEGF, anti-IL29, anti-NRG1-beta1 and anti-PD-ECGF, or any of its combinations, to perform a method according to any of clauses 4-14.

21.—A solid support, or DNA chip, that comprises oligonucleotides or single-channel microarrays designed from a known sequence or a mRNA of at least one of genes CD80, EG-VEGF, IL-29, NRG1-beta1 and PD-ECGF, or any of its combinations, to perform a method according to any of clauses 4-14.

22.—A computer readable storage medium that comprises software instructions that can lead a computer to perform the steps of the method according to any of clauses 4-14.

23.—A transmissible signal that comprises software instructions that can lead a computer to perform steps of the method according to any of clauses 4-14.

1. Simultaneous use of genes FGF-10, CXCL11, OSM, GPNMB and SCF, for the diagnosis of cancer of the pancreas.

2. The use, according to the above claim, wherein cancer of the pancreas is ductal adenocarcinoma of the pancreas (DACP).

3. A method for obtaining useful date for the diagnosis of cancer of the pancreas, that comprises:
a) obtaining an isolated sample from the individual.
b) measuring the amount of expression product of genes FGF-10, CXCL11, OSM, GPNMB and SCF.

4. The method according to the above claim, that also comprises:
c) comparing the amounts obtained in step (b) with a reference amount.

5. The method according to any of claims 3-4, where steps (b) and/or (c) can be completely or partially automated.

6. A diagnostic method for cancer of the pancreas that comprises steps (a)-(c) according to any of claims 3-5, and also comprises assigning the individual of step (a) to the group of individuals suffering cancer of the pancreas when they show an amount of expression products of genes FGF-10, CXCL11, OSM, GPNMB and SCF, higher than the reference amount.

7. The method according to any of claims 3-6, where cancer of the pancreas is ductal adenocarcinoma of the pancreas (DACP).

8. The method according to any of claims 3-7, wherein the biological sample is a blood sample, and preferably serum.

9. The method according to any of claims 3-8, wherein the measurement is performed by immunoassay.

10. The use of an antibody in the preparation of a medicine to treat an individual that suffers cancer of the pancreas, identifiable by a method according to any of claims 3-9, wherein the antibody is selected from anti-FGF-10, anti-CXCL11, anti-OSM, anti-GPNM and anti-SCF.

11. A pharmaceutical composition that comprises a modulatory agent of at least one of genes that are selected from FGF-10, CXCL11, OSM; GPNMB and SCF, in the preparation of a medicine for the treatment of an individual that suffers cancer of the pancreas, identifiable by a method according to any of claims 3-9.

12. A kit or device, that comprises the elements necessary to measure the amount of expression of genes that are selected from FGF-10, CXCL11, OSM, GPNMB and SCF.

13. The kit or device, according to claim 12, that comprises the antibodies anti-FGF-10, anti-CXCL11, anti-OSM, anti-GPNM and anti-SCF.

14. A solid support, or protein chip, that comprises at least one of the antibodies anti-FGF-10/, anti-CXCL11, anti-OSM, anti-GPNM and anti-SCF, or any of its combinations, to perform a method according to any of claims 3-9.

15. A solid support, or DNA chip, that comprises oligonucleotides or single-channel microarrays designed from a known sequence or a mRNA of at least one of genes FGF-10, CXCL11, OSM, GPNM and SCF, or any of its combinations, to perform a method according to any of claims 3-9.


SEQUENCE LISTING (32 sequences)

SEQ ID NO: 1 - DNA, 627 bp, Homo sapiens
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 2 - DNA, 1610 bp, Homo sapiens
agagaacaaaacagaaactcttggaagcaggaaaggtgcatgactcaaagagggaaattcctgtgccataaaaggattgctggtgtataaaatgctctatatatgccaattatcaatttcctttcatgttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 3 - DNA, 1869 bp, Homo sapiens
gtcacccccagcgggcgcgggccggagcacgggcacccagcatgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtagcctcgagagcaccccttgccggtgaaggatgcggcaggtgctctgtggatgagaggaaccatcgcaggatgacagctcccgggtccccaaacctgttcccctctgctactagccactgagaagtgcactttaagaggtgggagctgggcagacccctctacctcctccaggctgggagacagagtcaggctgttgcgctcccacctcagccccaagttccccaggcccagtggggtggccgggcgggccacgcgggaccgactttccattgattcaggggtctgatgacacaggctgactcatggccgggctgactgcccccctgccttgctccccgaggcctgccggtccttccctctcatgacttgcagggccgttgcccccagacttcctcctttccgtgtttctgaaggggaggtcacagcctgagctggcctcctatgcctcatcatgtcccaaaccagacacctggatgtctgggtgacctcactttaggcagctgtaacagcggcagggtgtcccaggagccctgatccgggggtccagggaatggagctcaggtcccaggccagccccgaagtcgccacgtggcctggggcaggtcactttacctctgtggacctgttttctctttgtgaagctagggagttagaggctgtacaaggcccccactgcctgtcggttgcttggattccctgacgtaaggtggatattaaaaatctgtaaatcaggacaggtggtgcaaatggcgctgggaggtgtacacggaggtctctgtaaaagcagacccacctcccagcgccgggaagcccgtcttgggtcctcgctgctggctgctccccctggtggtggatcctggaattttctcacgcaggagccattgctctcctagagggggtctcagaaactgcgaggccagttccttggagggacatgactaatttatcgatttttatcaatttttatcagttttatatttataagccttatttatgatgtatatttaatgttaatattgtgcaaacttatatttaaaacttgcctggtttctaaaaaaaaaaaaaaaaaa

SEQ ID NO: 4 - DNA, 2775 bp, Homo sapiens
tgccgcttaataccatcacatgatcctccccgaggccctgtatttaattaaaatagagagggaggcaccacagatgccagaagaacactgttgctcttggtggacgggcccagaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccgtatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggtcagtatttccagaaattgggacgatgttcagtgagagtttctgtgaacacagccaatgtgacacttgggcctcaactcatggaagtgactgtctacagaagacatggacgggcatatgttcccatcgcacaagtgaaagatgtgtacgtggtaacagatcagattcctgtgtttgtgactatgttccagaagaacgatcgaaattcatccgacgaaaccttcctcaaagatctccccattatgtttgatgtcctgattcatgatcctagccacttcctcaattattctaccattaactacaagtggagcttcggggataatactggcctgtttgtttccaccaatcatactgtgaatcacacgtatgtgctcaatggaaccttcagccttaacctcactgtgaaagctgcagcaccaggaccttgtccgccaccgccaccaccacccagaccttcaaaacccaccccttctttagcaactactctaaaatcttatgattcaaacaccccaggacctgctggtgacaaccccctggagctgagtaggattcctgatgaaaactgccagattaacagatatggccactttcaagccaccatcacaattgtagagggaatcttagaggttaacatcatccagatgacagacgtcctgatgccggtgccatggcctgaaagctccctaatagactttgtcgtgacctgccaagggagcattcccacggaggtctgtaccatcatttctgaccccacctgcgagatcacccagaacacagtctgcagccctgtggatgtggatgagatgtgtctgctgactgtgagacgaaccttcaatgggtctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatatttgtcactgtgatctccctcttggtgtacaaaaaacacaaggaatacaacccaatagaaaatagtcctgggaatgtggtcagaagcaaaggcctgagtgtctttctcaaccgtgcaaaagccgtgttcttcccgggaaaccaggaaaaggatccgctactcaaaaaccaagaatttaaaggagtttcttaaatttcgaccttgtttctgaagctcacttttcagtgccattgatgtgagatgtgctggagtggctattaacctttttttcctaaagattattgttaaatagatattgtggtttggggaagttgaattttttataggttaaatgtcattttagagatggggagagggattatactgcaggcagcttcagccatgttgtgaaactgataaaagcaacttagcaaggcttcttttcattattttttatgtttcacttataaagtcttaggtaactagtaggatagaaacactgtgtcccgagagtaaggagagaagctactattgattagagcctaacccaggttaactgcaagaagaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtacagtcgctgcacatatttgaaatcatatattaagactttccaaagatgaggtccctggtttttcatggcaacttgatcagtaaggatttcacctctgtttgtaactaaaaccatctactatatgttagacatgacattctttttctctccttcctgaaaaataaagtgtgggaagagacaagaaaaaaaaaa

SEQ ID NO: 5 - DNA, 2739 bp, Homo sapiens
tgccgcttaataccatcacatgatcctccccgaggccctgtatttaattaaaatagagagggaggcaccacagatgccagaagaacactgttgctcttggtggacgggcccagaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccgtatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggtcagtatttccagaaattgggacgatgttcagtgagagtttctgtgaacacagccaatgtgacacttgggcctcaactcatggaagtgactgtctacagaagacatggacgggcatatgttcccatcgcacaagtgaaagatgtgtacgtggtaacagatcagattcctgtgtttgtgactatgttccagaagaacgatcgaaattcatccgacgaaaccttcctcaaagatctccccattatgtttgatgtcctgattcatgatcctagccacttcctcaattattctaccattaactacaagtggagcttcggggataatactggcctgtttgtttccaccaatcatactgtgaatcacacgtatgtgctcaatggaaccttcagccttaacctcactgtgaaagctgcagcaccaggaccttgtccgccaccgccaccaccacccagaccttcaaaacccaccccttctttaggacctgctggtgacaaccccctggagctgagtaggattcctgatgaaaactgccagattaacagatatggccactttcaagccaccatcacaattgtagagggaatcttagaggttaacatcatccagatgacagacgtcctgatgccggtgccatggcctgaaagctccctaatagactttgtcgtgacctgccaagggagcattcccacggaggtctgtaccatcatttctgaccccacctgcgagatcacccagaacacagtctgcagccctgtggatgtggatgagatgtgtctgctgactgtgagacgaaccttcaatgggtctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatatttgtcactgtgatctccctcttggtgtacaaaaaacacaaggaatacaacccaatagaaaatagtcctgggaatgtggtcagaagcaaaggcctgagtgtctttctcaaccgtgcaaaagccgtgttcttcccgggaaaccaggaaaaggatccgctactcaaaaaccaagaatttaaaggagtttcttaaatttcgaccttgtttctgaagctcacttttcagtgccattgatgtgagatgtgctggagtggctattaacctttttttcctaaagattattgttaaatagatattgtggtttggggaagttgaattttttataggttaaatgtcattttagagatggggagagggattatactgcaggcagcttcagccatgttgtgaaactgataaaagcaacttagcaaggcttcttttcattattttttatgtttcacttataaagtcttaggtaactagtaggatagaaacactgtgtcccgagagtaaggagagaagctactattgattagagcctaacccaggttaactgcaagaagaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtacagtcgctgcacatatttgaaatcatatattaagactttccaaagatgaggtccctggtttttcatggcaacttgatcagtaaggatttcacctctgtttgtaactaaaaccatctactatatgttagacatgacattctttttctctccttcctgaaaaataaagtgtgggaagagacaagaaaaaaaaaa

SEQ ID NO: 6 - DNA, 5376 bp, Homo sapiens
gggcttcgctcgccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcagcctgtagttatcctggtctctgcaagtagatttcagcttggatagtgagggtaacaatttttctcaaagggatctggaaaaaatgtttaaaactcagtagtgtcagccactgtacagtgtagaaagcagtgggaactgtgattggatttggcaacatgtcagctttatagttgccgattagtgatatgggtctgatttcgatctcttcctgatgtaaaccatgctcacccatatcccactatacaaatgcaaatggttgcctggttccatttatgcaagggagccagtactgaattatgccttggcagaggggagactccaaaagagtcatcgcaggaagaagttaagaacactgaacatcagaacagtctgccaagaaggacattggcatcctgggaaagtccgccttttcccttgaccactatagggtgtataaatcgtgtttgcaaaatgtgttatgatgtgtttatattctaaaactattacagagctatgtaaagggacttaggagaaaatgctgaatgtaagatggtcccatttcaatttccaccatgggagagcctaaaaataaattatgacatttagtatctaaggttagaaaaccacgcccacatgctaatatgggtgttgaaaactaggttacttataatgcaaggaatcaggaaactttagttatttatagtataatcaccattatctgtttaaaggatccatttagttaaaatcgggcactctatattcattaaggtttatgaattaaaaagaaagctttatgtagttatgcatgtcagtttgctatttaaaatgtgtgacagtgtttgtcatattaagagtgaatttggcaggaattcccaagatggacattgtgcttttaaactagaacttgtaagacattatgtgaatatcccttgccaattttttttataataagaaaacatctgactaaagtcaaagaatgatttcttatggtttattttgatgaaagttcttttaacatgtcttgaatgtacacataaaggaatccaaagctttccattctaacttaatctttgtgataacattattgccatgttctacaaccgtaagatgacagttttcaatgtagtgacacaaaagggcatgaaaaactaactgctagctttcctttcatttcaaaagtccaagaatttctagtatatttggattttagcttctgttcaaagcaaatccagatgcaactccagtaagtggcctttgctcttttttgtaccaaagagcccagatgattcctacagtccctttcttctctaacatgctgtggttccttaaatatgagtaatttctctaagatataacccaggtgctttgagaagctgcattaaggtgttcaggccctcagatatcacatggtacacttgattagtaataaaaccagagatcaatttaaattgctgataggtcctgtctcagtgtgtggcattgactgttttcaggaaaatagatacagattaatatgagttatgcgtgtaggttgtgtatagattgagaagatagatacttctcaatctagtagtttgatttatttaaccaatggtttcagtttgcttgagcatatgaaaatcctgcttaatgtgcttaagagtataataaatgtgtacttttgtcctcaaacctagtagctgggttttaacactcatggacatggtcttaatcaatggagttaaataaacaaattcagcaagttattaaatctgacatggtaggagaggggagatgtgtcctgcttattaaatgtgttggtccattgaaagttacatggattgccaatttttaaaacactaaagttgaataaaatgcatgaacaatagaaaaatgctgaacattattttggatgctagctgcttggacattaactgtgttatttctgctttgagatgaaaatatatatttatctttgcttattttatcccagatgtgttctgaatatccttcttcataaatcatggaaaactcactgctgagatagtaaaccatgaaatcgccttttcagttggtgccatgtatctgacagttccatcttggaaggtttcaaaattaccttttaaaatgatctcagaagtctgtagattctcaatgatactgaaagctttgcacctctttggtagaaaccaggtctatttagaaaatggctttatgataaatgttgcctcctgagtgataatgaagtgttcctggatattgtattgtaatttaatgtgcttaccacactgccacattttaatgagtcagagaaaaattaatttttcttcaatacaataatagaacaagtagcctattctcttaaaaagtatgtgaaaagaaaattatgaaaaaatatgcatacctaatgaagtattggttttagtaagaattaaatacatttcattgagctttaaagtactttggagaaactttggggcacgttttcctactctaattcaactaaagttataaataaagagaaaaactcattcagaaatcatggattttaaaaatattttactgcagccaagttttcatttcaaaatgtaatttcagtttggagcttttaggcattatgtatatttaaaaaatatattcttcaaaaatgcattttggcatggtgggatggatgttgcaaaagatatccggagcctccagtctgtcattaactgatatggtaaatcacctctcttctttgggtctcaattttttatttatctatatggtaaactcagagatcactccttaggggtgagtcctattgcaatatgaccgacaaagaagacaaaatagcattgaaactaacccatacaaaatatccaactctggattctgtgaataagtatcttgaccataaaaagtcattgctgttcttgtttctaatgtaaatagtgtccattagtaaaagtgaaattcagtcttaagtagggtgaattggatcaccatttacacaagagatggctttttcctttgcttgaataaacattttggatcacctccaaagaatgaaaaccagtagtacgttttagtcatattagtcaggatgagaaactataagatgtgtgtaacatttggaaatgcaccaaagtgagcgtttaaatcttctcattttattgaaaactaagagcagaaaatgtaaaatgctcatgaaggttttgaatgccaaaagatattttagaatcaatttataaaggggtaattcattaattacactttaaaattggaaagtgggataagaaatctaaagtaaaccagcttatctttgaaacaatattattttgaaattggctttaaaataaaaccattcagattgaaattctaattagctcatttgtggagtttgatcacacaattcataatgttgctgctttccattaactagtcttgaaatgcctttgtttgtaaaaataaaataatggtactttcattttataacaaggtgtttttttcaagaaataatccatgctaaaatggatatttgtgatcctgaaatgtttactaagcattgtaaatttatttataactgccatctccaactacatccttatgatgtttttaacaataaaattaaaacaactgttaaactaaaaaccacaccgttttccagtacttgatctctgagctacaatactcactaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacaggtttgaatttaaaattacatgatatatacatatactttattgttctaaataaagaattttatgcactctcaaaaaaaaaaaaaaaaaa

SEQ ID NO: 7 - DNA, 5460 bp, Homo sapiens
gggcttcgctcgccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcagcctgtagttatcctggtctctgcaagtagatttcagcttggatagtgagggtaacaatttttctcaaagggatctggaaaaaatgtttaaaactcagtagtgtcagccactgtacagtgtagaaagcagtgggaactgtgattggatttggcaacatgtcagctttatagttgccgattagtgatatgggtctgatttcgatctcttcctgatgtaaaccatgctcacccatatcccactatacaaatgcaaatggttgcctggttccatttatgcaagggagccagtactgaattatgccttggcagaggggagactccaaaagagtcatcgcaggaagaagttaagaacactgaacatcagaacagtctgccaagaaggacattggcatcctgggaaagtccgccttttcccttgaccactatagggtgtataaatcgtgtttgcaaaatgtgttatgatgtgtttatattctaaaactattacagagctatgtaaagggacttaggagaaaatgctgaatgtaagatggtcccatttcaatttccaccatgggagagcctaaaaataaattatgacatttagtatctaaggttagaaaaccacgcccacatgctaatatgggtgttgaaaactaggttacttataatgcaaggaatcaggaaactttagttatttatagtataatcaccattatctgtttaaaggatccatttagttaaaatcgggcactctatattcattaaggtttatgaattaaaaagaaagctttatgtagttatgcatgtcagtttgctatttaaaatgtgtgacagtgtttgtcatattaagagtgaatttggcaggaattcccaagatggacattgtgcttttaaactagaacttgtaagacattatgtgaatatcccttgccaattttttttataataagaaaacatctgactaaagtcaaagaatgatttcttatggtttattttgatgaaagttcttttaacatgtcttgaatgtacacataaaggaatccaaagctttccattctaacttaatctttgtgataacattattgccatgttctacaaccgtaagatgacagttttcaatgtagtgacacaaaagggcatgaaaaactaactgctagctttcctttcatttcaaaagtccaagaatttctagtatatttggattttagcttctgttcaaagcaaatccagatgcaactccagtaagtggcctttgctcttttttgtaccaaagagcccagatgattcctacagtccctttcttctctaacatgctgtggttccttaaatatgagtaatttctctaagatataacccaggtgctttgagaagctgcattaaggtgttcaggccctcagatatcacatggtacacttgattagtaataaaaccagagatcaatttaaattgctgataggtcctgtctcagtgtgtggcattgactgttttcaggaaaatagatacagattaatatgagttatgcgtgtaggttgtgtatagattgagaagatagatacttctcaatctagtagtttgatttatttaaccaatggtttcagtttgcttgagcatatgaaaatcctgcttaatgtgcttaagagtataataaatgtgtacttttgtcctcaaacctagtagctgggttttaacactcatggacatggtcttaatcaatggagttaaataaacaaattcagcaagttattaaatctgacatggtaggagaggggagatgtgtcctgcttattaaatgtgttggtccattgaaagttacatggattgccaatttttaaaacactaaagttgaataaaatgcatgaacaatagaaaaatgctgaacattattttggatgctagctgcttggacattaactgtgttatttctgctttgagatgaaaatatatatttatctttgcttattttatcccagatgtgttctgaatatccttcttcataaatcatggaaaactcactgctgagatagtaaaccatgaaatcgccttttcagttggtgccatgtatctgacagttccatcttggaaggtttcaaaattaccttttaaaatgatctcagaagtctgtagattctcaatgatactgaaagctttgcacctctttggtagaaaccaggtctatttagaaaatggctttatgataaatgttgcctcctgagtgataatgaagtgttcctggatattgtattgtaatttaatgtgcttaccacactgccacattttaatgagtcagagaaaaattaatttttcttcaatacaataatagaacaagtagcctattctcttaaaaagtatgtgaaaagaaaattatgaaaaaatatgcatacctaatgaagtattggttttagtaagaattaaatacatttcattgagctttaaagtactttggagaaactttggggcacgttttcctactctaattcaactaaagttataaataaagagaaaaactcattcagaaatcatggattttaaaaatattttactgcagccaagttttcatttcaaaatgtaatttcagtttggagcttttaggcattatgtatatttaaaaaatatattcttcaaaaatgcattttggcatggtgggatggatgttgcaaaagatatccggagcctccagtctgtcattaactgatatggtaaatcacctctcttctttgggtctcaattttttatttatctatatggtaaactcagagatcactccttaggggtgagtcctattgcaatatgaccgacaaagaagacaaaatagcattgaaactaacccatacaaaatatccaactctggattctgtgaataagtatcttgaccataaaaagtcattgctgttcttgtttctaatgtaaatagtgtccattagtaaaagtgaaattcagtcttaagtagggtgaattggatcaccatttacacaagagatggctttttcctttgcttgaataaacattttggatcacctccaaagaatgaaaaccagtagtacgttttagtcatattagtcaggatgagaaactataagatgtgtgtaacatttggaaatgcaccaaagtgagcgtttaaatcttctcattttattgaaaactaagagcagaaaatgtaaaatgctcatgaaggttttgaatgccaaaagatattttagaatcaatttataaaggggtaattcattaattacactttaaaattggaaagtgggataagaaatctaaagtaaaccagcttatctttgaaacaatattattttgaaattggctttaaaataaaaccattcagattgaaattctaattagctcatttgtggagtttgatcacacaattcataatgttgctgctttccattaactagtcttgaaatgcctttgtttgtaaaaataaaataatggtactttcattttataacaaggtgtttttttcaagaaataatccatgctaaaatggatatttgtgatcctgaaatgtttactaagcattgtaaatttatttataactgccatctccaactacatccttatgatgtttttaacaataaaattaaaacaactgttaaactaaaaaccacaccgttttccagtacttgatctctgagctacaatactcactaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacaggtttgaatttaaaattacatgatatatacatatactttattgttctaaataaagaattttatgcactctcaaaaaaaaaaaaaaaaaa

SEQ ID NO: 8 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 9 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 10 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 11 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 12 - protein, 560 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 13 - protein, 245 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKGKAKNPPGDSSLHWAAMALPALFSLIIGFAFGALYWKKRQPSLTRAVENIQINEEDNEISMLQEKEREFQEV

SEQ ID NO: 14 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 15 - DNA, 2757 bp, Homo sapiens
gacaagtactgagtgaactcaaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtcccacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggtgttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataa

SEQ ID NO: 16 - DNA, 1372 bp, Homo sapiens
gaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttcctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacctgctggctttagaataaaagcaccaactgaa

SEQ ID NO: 17 - DNA, 856 bp, Homo sapiens
aattaccttttcactttacacacatcatcttggattgcccattttgcgtggctaaaaagcagagccatgccgctggggaagcagttgcgatttagccatggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatgaagctgcagccctgactgagacatagggctgagtttattgttttacttttatacattatgcacaaataaacaacaaggaattgga

SEQ ID NO: 18 - DNA, 2553 bp, Homo sapiens
gcactcggggcgacagagagggaggaggcgcgcggggacggggacgcccaggaggacccactcgcgggtcccgctccgctccggcagcagcatggggaaaggacgcgcgggccgagttggcaccacagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaact

SEQ ID NO: 19 - DNA, 1667 bp, Homo sapiens
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaa

SEQ ID NO: 20 - DNA, 1682 bp, Homo sapiens
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatgcccctctccccgcaggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaa

SEQ ID NO: 21 - DNA, 1679 bp, Homo sapiens
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaa

SEQ ID NO: 22 - DNA, 1718 bp, Homo sapiens
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggccgcagaccccggacgtgtcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaaaaaaaaaaaaaaaaaa

SEQ ID NO: 23 - DNA, 1851 bp, Homo sapiens
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggtacgggcggggctcaagtcgcgaggcggggaagcgggaggcagacacggacgagggcgacacagacacgggaccgaggggcggacaccggagagacacgggaaaggggtcgggacaggagcacgtggctcagacaccgacgccgggaggccgcagaccccggacgtgtcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaa

SEQ ID NO: 24 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 25 - protein, 105 aa, Homo sapiens
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 26 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 27 - protein, 640 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 28 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 29 - protein, 487 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADAPLPAGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 30 - protein, 482 aa, Homo sapiens; identical to SEQ ID NO: 28

SEQ ID NO: 31 - protein, 482 aa, Homo sapiens; identical to SEQ ID NO: 28

SEQ ID NO: 32 - protein, 482 aa, Homo sapiens; identical to SEQ ID NO: 28